(19) United States
(12) Patent Application Publication
(10) Pub. No.: US 2022/0170918 A1
(43) Pub. Date: Jun. 2, 2022

(54) SUBSTRATE FOR SINGLE MOLECULE ORGANIZATION

(71) Applicant: Palamedrix, Inc., La Jolla, CA (US)

(72) Inventors: Ashwin Gopinath, Palo Alto, CA (US); Paul Rothemund, Pasadena, CA (US); Rishabh Shetty, San Diego, CA (US); Shane Bowen, Encinitas, CA (US)

(21) Appl. No.: 17/537,004

(22) Filed: Nov. 29, 2021

Related U.S. Application Data
(60) Provisional application No. 63/119,316, filed on Nov. 30, 2020.

(51) Int. Cl. G01N 33/53; G01N 27/414

(52) U.S. Cl. CPC G01N 33/5308; G01N 27/4145

(57) ABSTRACT

Provided herein are structures and methods for detecting one or more analyte molecules present in a sample. In some embodiments, the one or more analyte molecules are detected using one or more supramolecular structures that are coupled to a substrate, e.g., a solid support. In some embodiments, the supramolecular structures are bi-stable, wherein the supramolecular structures transition from an unstable state to a stable state through interaction with one or more analyte molecules from the sample. In some embodiments, the stable state supramolecular structures are configured to provide a signal for analyte molecule detection and quantification.

### CROSS-REFERENCE TO RELATED APPLICATIONS

The present application claims priority to and the benefit of U.S. Provisional Application No. 63/119,316, filed Nov. 30, 2020, and entitled SUBSTRATE FOR SINGLE MOLECULE ORGANIZATION, the disclosure of which is hereby incorporated by reference herein in its entirety.

### BACKGROUND

The current state of personalized healthcare is overwhelmingly genome-centric, predominantly focused on quantifying the genes present within an individual. While such an approach has proven to be extremely powerful, it does not provide a clinician with the complete picture of an individual's health. This is because genes are the “blueprints” of an individual and it merely informs the likelihood of developing an ailment. Within an individual these “blueprints” first need to be transcribed into RNA and then translated into various protein molecules, the real “actors” in the cell, in order to have any effect on the health of an individual.

The concentration of proteins, the interaction between the proteins (protein-protein interactions or PPI), as well as the interaction between proteins and small molecules, are intricately linked to the health of different organs, homeostatic regulatory mechanism as well as the interaction of these systems with the external environment. Hence, quantitative information about proteins as well as PPIs is vital to create a complete picture of an individual's health at a given time point as well as to predict any emerging health issues. For instance, the amount of stress experienced by cardiac muscles (e.g. during a heart attack) can be inferred by measuring the concentration of troponin I/II and myosin light chain present within peripheral blood. Similar protein biomarkers have also been identified, validated and are deployed for a wide variety of organ dysfunctions (e.g. liver disease and thyroid disorders), specific cancers (e.g. colorectal or prostate cancer), and infectious diseases (e.g. HIV and Zika). The interaction between these proteins are also essential for drug development and are increasingly becoming a highly sought-after dataset. The ability to detect and quantify proteins and other molecules, within a given sample of bodily fluids, is an integral component of such healthcare development.

### SUMMARY

The present disclosure generally relates to systems, structures and methods for detection and quantification of analyte molecules in a sample.

Provided herein, in some embodiments, is a method for detecting an analyte molecule present in a sample, the method comprising: a) providing a solid support; b) patterning the solid support to form a plurality of binding sites on the solid support; and c) associating a single supramolecular structure with a binding site from among the plurality of binding sites on the solid support, wherein the supramolecular structure comprises: i) a core structure comprising a plurality of core molecules, ii) a capture molecule linked to the core structure at a first location, and iii) a detector molecule linked to the core structure at a second location, wherein the supramolecular structure is in an unstable state, such that the detector molecule is configured to be unbound from the core structure through cleavage of a link therebetween at the second location.

Provided herein, in some embodiments, is a method for detecting an analyte molecule using a substrate, the method comprising: providing the substrate, the substrate comprising a plurality of binding sites, wherein an individual binding site of the plurality of binding sites is associated with a supramolecular structure of a plurality of supramolecular structures, the supramolecular structure comprising: a core structure comprising a plurality of core molecules a capture molecule linked to the core structure at a first location, and a detector molecule linked to the core structure at a second location, wherein the supramolecular structure is in an unstable state, such that the detector molecule is configured to be unbound from the core structure through cleavage of a link therebetween at the second location; contacting the sample with the supramolecular structure, such that the supramolecular structure shifts from the unstable state to a stable state wherein the detector molecule and the capture molecule are linked together through binding to the analyte molecule, thereby forming a link between the detector molecule and capture molecule and wherein each supramolecular structure in the unstable state comprises the respective capture molecule and detector molecule spaced apart at a pre-determined distance; providing a trigger to cleave the link between the detector molecule and the core structure at the second location, wherein the detector molecule remains linked to the core structure through the link with the capture molecule and such that the analyte molecule is associated with the individual binding site; and detecting the analyte molecule based on a signal provided by the supramolecular structure that shifted to the stable state. In other embodiment, the supramolecular structure does not include an integral detector molecule, and the detection is mediated through a separate step.

Provided herein, in some embodiments, is a method for forming a substrate for detection of an analyte molecule in a sample. The method includes the steps of providing a base layer; providing a binding layer on the base layer; depositing a top layer on the binding layer; patterning the top layer to expose portions of the binding layer, the exposed portions corresponding to a plurality of binding sites on the binding layer; and providing a supramolecular structure associated with each binding site of the plurality of binding sites, the supramolecular structure comprising: a core structure comprising a plurality of core molecules a capture molecule linked to the core structure at a first location, and a detector molecule linked to the core structure at a second location, wherein the supramolecular structure is in an unstable state, such that the detector molecule is configured to be unbound from the core structure through cleavage of a link therebetween at the second location and such that the respective capture molecule and detector molecule spaced apart are at a pre-determined distance in the unstable state and wherein, in a stable state, the detector molecule and the capture molecule are linked together through binding to the analyte molecule, thereby forming a link between the detector molecule and capture molecule, wherein the detector molecule remains linked to the core structure through the link with the capture molecule and such that the analyte molecule is associated with the individual binding site.

In some embodiments, any method disclosed herein further comprising quantifying the concentration of the analyte molecule in the sample. In some embodiments, any method disclosed herein further comprising identifying the detected analyte molecule. In some embodiments, any method disclosed herein further comprising detecting the analyte molecule based on the signal when the analyte molecule is present in the sample at a count of a single molecule or higher. In some embodiments, the solid support is coupled to or associated with a detection system that generates a signal based on a detectable change indicative of the presence of or formation of a stable state, as a result of analyte capture or binding by the supramolecular structure, at an individual binding site.

In some embodiments, for any method disclosed herein, the sample comprises a complex biological sample and the method provides for single-molecule sensitivity thereby increasing a dynamic range and quantitative capture of a range of molecular concentrations within the complex biological sample. In some embodiments, for any method disclosed herein, the analyte molecule comprises a protein, a peptide, a peptide fragment, a lipid, a DNA, a RNA, an organic molecule, an inorganic molecule, complexes thereof, or any combinations thereof. In some embodiments, for any method disclosed herein, each supramolecular structure is a nanostructure.

In some embodiments, for any method disclosed herein, each core structure is a nanostructure. In some embodiments, for any method disclosed herein, the plurality of core molecules for each core structure are arranged into a pre-defined shape and/or have a prescribed molecular weight. In some embodiments, the pre-defined shape is configured to limit or prevent cross-reactivity with another supramolecular structure. In some embodiments, for any method disclosed herein, the plurality of core molecules for each core structure comprises one or more nucleic acid strands, one or more branched nucleic acids, one or more peptides, one or more small molecules, or combinations thereof. In some embodiments, for any method disclosed herein, each core structure independently comprises a scaffolded deoxyribonucleic acid (DNA) origami, a scaffolded ribonucleic acid (RNA) origami, a scaffolded hybrid DNA:RNA origami, a single-stranded DNA tile structure, a multi-stranded DNA tile structure, a single-stranded RNA origami, a multi-stranded RNA tile structure, hierarchically composed DNA or RNA origami with multiple scaffolds, a peptide structure, or combinations thereof.

In some embodiments, for any method disclosed herein, the trigger comprises a deconstructor molecule, a trigger signal, or combinations thereof. In some embodiments, the deconstructor molecule comprises DNA, RNA, a peptide, a small organic molecule, or combinations thereof. In some embodiments, the trigger signal comprises an optical signal, an electrical signal, or both. In some embodiments, the trigger optical signal comprises a microwave signal, an ultraviolet illumination, a visible illumination, a near infrared illumination, or combinations thereof.

In some embodiments, for any method disclosed herein, the respective analyte molecule is 1) bound to the capture molecule of the respective supramolecular structure through a chemical bond and/or 2) bound to the detector molecule of the respective supramolecular structure through a chemical bond. In some embodiments, for any method disclosed herein, the capture molecule and detector molecule for each supramolecular structure independently comprise a protein, a peptide, an antibody, an aptamer (RNA and DNA), a fluorophore, a darpin, a catalyst, a polymerization initiator, a polymer like PEG, or combinations thereof. In some embodiments, for any method disclosed herein, wherein for each supramolecular structure: a) the capture molecule is linked to the core structure through a capture barcode, wherein the capture barcode comprises a first capture linker, a second capture linker, and a capture bridge disposed between the first and second capture linkers, wherein the first capture linker is bound to a first core linker that is bound to the first location on the core structure, wherein the capture molecule and the second capture linker are linked together through binding to a third capture linker, and b) the detector molecule is linked to the core structure through a detector barcode, wherein the detector barcode comprises a first detector linker, a second detector linker, and a detector bridge disposed between the first and second detector linkers, wherein the first detector linker is bound to a second core linker that is bound to the second location on the core structure, wherein the detector molecule and the second detector linker are linked together through binding to a third detector linker. In some embodiments, the capture bridge and detector bridge independently comprise a polymer core. In some embodiments, the polymer core of the capture bridge and the polymer core of the detector bridge independently comprise a nucleic acid (DNA or RNA) of specific sequence or a polymer like PEG. In some embodiments, the first core linker, second core linker, first capture linker, second capture linker, third capture linker, first detector linker, second detector linker, and third detector linker independently comprise a reactive molecule or DNA sequence domain. In some embodiments, each reactive molecule independently comprises an amine, a thiol, a DBCO, a maleimide, biotin, an azide, an acrydite, a NETS-ester, a single stranded nucleic acid (RNA or DNA) of specific sequence, one or more polymers like PEG or polymerization initiators, or combinations thereof. In some embodiments, the linkage between the capture barcode and 1) the first core linker, and/or 2) the third capture linker comprises a chemical bond. In some embodiments, the chemical bond comprises a covalent bond. In some embodiments, the linkage between the detector barcode and 1) the second core linker, and/or 2) the third detector linker comprises a chemical bond. In some embodiments, the chemical bond comprises a covalent bond. In some embodiments, the trigger cleaves the linkage between 1) the first detector linker and the second core linker and/or 2) the first capture linker and the first core linker. In some embodiments, for any method disclosed herein, the capture molecule is bound to the third capture linker through a chemical bond and/or the detector molecule is bound to the third detector linker through a chemical bond. In some embodiments, the capture molecule is covalently bonded to the third capture linker and/or the detector molecule is covalently bonded to the third detector linker.

In some embodiments, for any method disclosed herein, each supramolecular structure in the unstable state comprises the respective capture molecule and detector molecule spaced apart at a pre-determined distance, so as to reduce or inhibit the occurrence of cross-reactions between capture and/or detector molecules of a first supramolecular structure and corresponding capture and/or detector molecules of a second supramolecular structure. In some embodiments, for any method disclosed herein, the pre-determined distance is from about 3 nm to about 40 nm.

In some embodiments, for any method disclosed herein, each supramolecular structure further comprises an anchor molecule linked to the core structure. In some embodiments, the anchor molecule is linked to the core structure via an anchor barcode, wherein the anchor barcode comprises a first anchor linker, a second anchor linker, and an anchor bridge disposed between the first and second anchor linkers, wherein the first anchor linker is bound to a third core linker that is bound to a third location on the core structure, wherein the anchor molecule is linked to the second anchor linker. In some embodiments, the anchor molecule comprises an amine, a thiol, a DBCO, a maleimide, biotin, an azide, an acrydite, a NETS-ester, a single stranded nucleic acid (RNA or DNA) of specific sequence, one or more polymers like PEG or polymerization initiators, or combinations thereof. In some embodiments, the anchor bridge comprises a polymer core. In some embodiments, the polymer core of the anchor bridge comprises a nucleic acid (DNA or RNA) of specific sequence or a polymer like PEG. In some embodiments, the third core linker, first anchor linker, second anchor linker, and anchor molecule independently comprise an anchor reactive molecule or DNA sequence domain. In some embodiments, each anchor reactive molecule independently comprises an amine, a thiol, a DBCO, a maleimide, biotin, an azide, an acrydite, a NETS-ester, a single stranded nucleic acid (RNA or DNA) of specific sequence, one or more polymers like PEG or polymerization initiators, or combinations thereof. In some embodiments, the anchor molecule is linked to the second anchor linker through a chemical bond. In some embodiments, the anchor molecule is covalently bonded to the second anchor linker. In some embodiments, wherein the trigger further cleaves 1) the second anchor linker from the anchor molecule, 2) the first anchor linker from the third core linker, or combinations thereof. In some embodiments, the first and second locations are situated on a first side of the core structure, and the third location is situated on a second side of the core structure.

In some embodiments, for any method disclosed herein, the signal comprises the detector barcode, the capture barcode, or combinations thereof, corresponding to a supramolecular structure that shifted to a stable state. In some embodiments, any method disclosed herein, further comprising separating each detector barcode from a corresponding detector molecule for the at least one supramolecular structure that shifted to a stable state, such that the corresponding signal comprises the respective detector barcode for detection of the analyte molecule bound to the respective capture and detector molecules. In some embodiments, each separated detector barcode provides a DNA signal corresponding to the analyte molecule bound to the respective detector molecule. In some embodiments, the at least one separated detector barcodes are analyzed using genotyping, qPCR, sequencing, or combinations thereof. In some embodiments, a plurality of analyte molecules in the sample are detected simultaneously through multiplexing via one or more supramolecular structures that shifted to a stable state. In some embodiments, for any method disclosed herein, the capture and detector molecules for each supramolecular structure is configured for binding to one or more specific types of analyte molecules.

In some embodiments, for any method comprising using a plurality of supramolecular structures disclosed herein, each core structure of the plurality of supramolecular structures are identical to each other. In some embodiments, each supramolecular structure comprises a prescribed shape, size, molecular weight, or combinations thereof, so as to reduce or eliminate cross-reactions between a plurality of supramolecular structures. In some embodiments, each supramolecular structure comprises a plurality of capture and detector molecules. In some embodiments, each supramolecular structure comprises a prescribed stoichiometry of the capture and detector molecules so as to reduce or eliminate cross-reactions between the plurality of supramolecular structures.

In some embodiments, for any method comprising using a plurality of supramolecular structures disclosed herein, the unstable state for each supramolecular structure further comprises the capture and detector molecules spaced apart at a pre-determined distance so as to reduce or inhibit the occurrence of cross-reactions between capture and/or detector molecules of a first supramolecular structure and a second supramolecular structure. In some embodiments, the pre-determined distance is from about 3 nm and about 40 nm. In some embodiments, the mean distance between any two supramolecular structures is larger than the pre-determined distance between the capture and detector molecules of a respective supramolecular structure. In some embodiments, the mean distance between any two supramolecular structures is larger than the pre-determined distance between the capture and detector molecules of a respective supramolecular structure. In some embodiments, the plurality of supramolecular structures are attached to one or more solid substrates. In some embodiments, each solid substrate of the one or more solid substrates comprises a planar substrate and/or comprises a planar substrate having a patterned or shaped surface having an array of binding sites. In some embodiments, a plurality of supramolecular structures are disposed on the substrate, wherein the substrate comprises a plurality of binding sites, wherein each binding site is associated with a corresponding supramolecular structure. In an embodiment, an individual binding site of an array of binding sites of a substrate is associated with at most one supramolecular structure, e.g., only one supramolecular structure per binding site. In some embodiments, the plurality of supramolecular structures distributed on a substrate are configured to detect the same analyte molecule. In some embodiments, a plurality of signaling elements are configured to link with the detector molecules of the at least one supramolecular structure that shifted to the stable state. In some embodiments, each signaling element comprises a fluorescent molecule or microbead, a fluorescent polymer, highly charged nanoparticles or polymer. In some embodiments, at least one supramolecular structure of the plurality of supramolecular structures on a substrate is configured to detect a different analyte molecule from at least one of the other supramolecular structures. In some embodiments, an individual supramolecular structure is barcoded with unique barcodes to distinguish each supramolecular structure on the substrate from one another so as to identify the location of each supramolecular structure on the substrate. In some embodiments a plurality of signaling elements link with the detector molecules of the at least one supramolecular structure that shifted to the stable state. In some embodiments, each signaling element comprises a fluorescent molecule or microbead, a fluorescent polymer, highly charged nanoparticles or polymer.

Provided herein is method for forming a substrate, the method comprising: providing a base layer; providing a binding layer on the base layer; depositing a top layer on the binding layer; patterning the top layer to expose portions of the binding layer, the exposed portions corresponding to a plurality of binding sites on the binding layer; and associating a molecule or structure at each binding site. In an embodiment, the method includes providing a supramolecular structure associated with each binding site of the plurality of binding sites, the supramolecular structure comprising a core structure, the core structure comprising a plurality of core molecules. The core structure may be linked to one or more cargo molecules that have affinity for or that bind to an analyte. The present techniques may be generally used for organizing single molecules, e.g., one or more core structures of a supramolecular structure, and can thus find use in a variety of different areas. In an embodiment, present techniques may be generally used for organizing a single quantum dot.

In some embodiments, for any method disclose herein, additional steps can be performed to regenerate a formed substrate after analyte detection is complete. Regeneration may include removing all or a portion of the supramolecular structure.

In some embodiments, an analyte or analyte molecule in a sample is detected. The analyte may comprises a biological particle or a biomolecule. In some embodiments, the analyte molecule comprises a chemical compound, a protein, a peptide, a fragment of a peptide, a lipid, a nucleic acid, DNA, RNA, an organic molecule, a viral particle, an exosome, an organelle, or any complexes thereof. In some embodiments, the sample comprises a tissue biopsy, blood, blood plasma, Urine, Saliva, Tear, Cerebrospinal fluid, extracellular fluid, cultures cells, culture media, discarded tissue, plant matter, a synthetic protein, a bacterial and/or viral sample or fungal tissue, or combinations thereof.

Provided herein, in some embodiments, is a substrate for detecting one or more analyte molecules in a sample, the substrate comprising a plurality of binding sites associated with a respective plurality of supramolecular structures, each supramolecular structure comprising: a) a core structure comprising a plurality of core molecules, b) a capture molecule linked to the supramolecular core at a first location, and c) a detector molecule linked to the supramolecular core at a second location, wherein the supramolecular structure is in an unstable state, such that the detector molecule is configured to be unbound from the core structure through cleavage of a link therebetween at the second location; wherein each supramolecular structure is configured to shift from the unstable state to a stable state through interaction between the detector molecule, the capture molecule, and a respective analyte molecule of the one or more analyte molecules; wherein, upon interaction with a trigger, a respective supramolecular structure that shifted to the stable state provides a signal for detecting the respective analyte molecule.

Provided herein, in some embodiments, is a substrate for detecting one or more analyte molecules in a sample, the substrate comprising: a base layer; a binding layer on the base layer; a patterned top layer exposing portions of the binding layer, the exposed portions corresponding to a plurality of binding sites on the binding layer; and a supramolecular structure associated with each binding site of the plurality of binding sites. The supramolecular structure comprises a core structure comprising a plurality of core molecules, a capture molecule linked to the supramolecular core at a first location, and a detector molecule linked to the supramolecular core at a second location, wherein the supramolecular structure is in an unstable state, such that the detector molecule is configured to be unbound from the core structure through cleavage of a link therebetween at the second location and wherein each supramolecular structure is configured to shift from the unstable state to a stable state through interaction between the detector molecule, the capture molecule, and a respective analyte molecule of the one or more analyte molecules.

In some embodiments, wherein upon interaction with the trigger, each detection molecule linked to a supramolecular structure in the unstable state becomes unbound from said supramolecular structure. In some embodiments, each core structure of the plurality of supramolecular structures is identical to each other. In some embodiments, the mean distance between any two supramolecular structures is larger than the pre-determined distance between the capture and detector molecules of a respective supramolecular structure. In some embodiments, the substrate comprises a solid support or a solid substrate.

In some embodiments, each core structure of the plurality of supramolecular structures are identical to each other. In some embodiments, each supramolecular structure comprises a prescribed shape, size, molecular weight, or combinations thereof, so as to reduce or eliminate cross-reactions between a plurality of supramolecular structures. In some embodiments, each supramolecular structure comprises a plurality of capture and detector molecules. In some embodiments, each supramolecular structure comprises a prescribed stoichiometry of the capture and detector molecules so as to reduce or eliminate cross-reactions between the plurality of supramolecular structures. In some embodiments, the unstable state for each supramolecular structure further comprises the capture and detector molecules spaced apart at a pre-determined distance so as to reduce or inhibit the occurrence of cross-reactions between capture and/or detector molecules of a first supramolecular structure and a second supramolecular structure. In some embodiments, the pre-determined distance is from about 3 nm and about 40 nm. In some embodiments, the mean distance between any two supramolecular structures is larger than the pre-determined distance between the capture and detector molecules of a respective supramolecular structure.

In some embodiments, each substrate comprises a widget, a solid support, a polymer matrix, a solid substrate, or a molecular condensate. In some embodiments, the mean distance between any two supramolecular structures is larger than the pre-determined distance between the capture and detector molecules of a respective supramolecular structure. In some embodiments, the solid substrate comprises a planar substrate. In some embodiments, a plurality of supramolecular structures are disposed on the substrate, wherein the substrate comprises a plurality of binding sites, wherein each binding site is configured to link with a corresponding supramolecular structure. In some embodiments, the plurality of supramolecular structures are configured to detect the same analyte molecule. In some embodiments, a plurality of signaling elements are configured to link with the detector molecules of the at least one supramolecular structure that shifted to the stable state. In some embodiments, each signaling element comprises a fluorescent molecule or microbead, a fluorescent polymer, highly charged nanoparticles or polymer. In some embodiments, at least one supramolecular structure of the plurality of supramolecular structures is configured to detect a different analyte molecule from the other supramolecular structures.

In some embodiments, the supramolecular structure comprises a prescribed shape, size, molecular weight, or combinations thereof, so as to reduce or eliminate cross-reactions with another supramolecular structure and/or to align with a size and shape of a binding site in the substrate. In one example, the dimensions of the supramolecular structure in an x-y plane are selected to conform to or overlap a binding site size and shape. To encourage the association of only one supramolecular structure with one binding site, the binding site and the supramolecular structure may be sized and shaped such that two supramolecular structures would not fit on a single binding site. In an embodiment, the supramolecular structure may be smaller than an individual binding site in at least one dimension when associated with the binding site. In some embodiments, the supramolecular structure comprises a plurality of capture and detector molecules. In some embodiments, the supramolecular structure comprises a prescribed stoichiometry of the capture and detector molecules so as to reduce or eliminate cross-reactions with another supramolecular structure.

### BRIEF DESCRIPTION OF THE DRAWINGS

Specific embodiments of the disclosed devices, delivery systems, or methods will now be described with reference to the drawings. Nothing in this detailed description is intended to imply that any particular component, feature, or step is essential to the invention.

FIG. 1A depicts an exemplary supramolecular structure and the related subcomponents.

FIG. 1B depicts an exemplary supramolecular structure and the related subcomponents.

FIG. 2 depicts an exemplary assembled three-arm nucleic acid junction based supramolecular structure and related subcomponents.

FIG. 3 depicts exemplary individual subcomponents of the three-arm nucleic acid junction based supramolecular structure from FIG. 2.

FIG. 4 depicts exemplary deconstructor molecules corresponding to the subcomponents of the three-arm nucleic acid junction based supramolecular structure from FIG. 2.

FIG. 5 depicts an exemplary assembled DNA origami based supramolecular structure and related subcomponents.

FIG. 6 depicts exemplary individual subcomponents of the DNA origami based supramolecular structure from FIG. 5.

FIG. 7 depicts exemplary deconstructor molecules corresponding to the subcomponents of the DNA origami based supramolecular structure from FIG. 5.

FIG. 8 provides an exemplary depiction of a supramolecular structure in an unstable state before and after being subject to a trigger (e.g., interaction with a deconstructor molecule).

FIG. 9 provides an exemplary depiction of a supramolecular structure in a stable state before and after being subject to a trigger (e.g., interaction with a deconstructor molecule).

FIG. 10 provides an exemplary depiction of a supramolecular structure shifting from an unstable state to a stable state after interaction with an analyte molecule, and the respective configurations before and after being subject to a trigger (e.g., interaction with a deconstructor molecule).

FIG. 11 provides an exemplary depiction of a supramolecular structure shifting from a stable state to an unstable state after interaction with an analyte molecule, and the respective configurations before and after being subject to a trigger (e.g., interaction with a deconstructor molecule).

FIG. 12 provides an exemplary depiction of a method for detecting and quantifying analyte molecules using a plurality of supramolecular structures.

FIG. 13 provides an exemplary depiction of a method for detecting and quantifying analyte molecules using a plurality of supramolecular structures attached to a planar substrate.

FIG. 14 provides an exemplary depiction of a technique for forming an array of binding sites for supramolecular structures that include DNA origami on a substrate.

FIG. 15 provides an exemplary depiction of a technique for forming an array of binding sites for supramolecular structures on a substrate.

FIG. 16 provides an exemplary depiction of a technique for forming an array of binding sites for supramolecular structures on a substrate.

FIG. 17 provides an exemplary depiction of a technique for forming an array of binding sites for supramolecular structures on a substrate.

FIG. 18 provides an exemplary depiction of a substrate coupled to a detection system.

FIG. 19 provides an exemplary depiction of a substrate coupled to a field-effect transistor detection system.

FIG. 20 provides an exemplary depiction of a substrate coupled to an optical detection system.

FIG. 21 provides an exemplary depiction of a workflow for regenerating a substrate.

FIG. 22 provides an exemplary depiction of a technique for forming an array of binding sites for supramolecular structures that include DNA origami on a substrate.

FIG. 23 provides an exemplary depiction of a technique for forming an array of binding sites for supramolecular structures that include DNA origami on a substrate.

FIG. 24 provides an exemplary depiction of a technique for forming an array of binding sites for supramolecular structures that include DNA origami on a substrate.

### DETAILED DESCRIPTION

Disclosed herein are structures and methods for detecting one or more analyte molecules present in a sample. In some embodiments, the one or more analyte molecules are detected using one or more supramolecular structures that are coupled to a substrate, e.g., a solid support. The solid support may include a plurality of binding sites that receive, at an individual binding site, a supramolecular structure. Thus, the substrate provides single molecule organization for a supramolecular structure. In one example, each binding site of the plurality of binding sites is associated with a single supramolecular structure. Analytes may be detected at one or more binding sites of the plurality of binding sites. Based on the patterning and placement of the supramolecular structures, and the characteristics, e.g., components, of each supramolecular structure on the substrate, an array with desired analytical functionality may be generated.

In some embodiments, the one or more supramolecular structures are specifically designed to minimize cross-reactivity with each other. In some embodiments, the supramolecular structures are bi-stable, wherein the supramolecular structures shift from an unstable state to a stable state through interaction with one or more analyte molecules from the sample. In some embodiments, the stable state supramolecular structures, as a result of transitioning to the stable state, are configured to provide a signal for analyte molecule detection and/or quantification. In some embodiments, the signal is an electrical signal, an optical signal, an electromagnetic signal, or a DNA signal, such that detection and quantification of an analyte molecule comprises converting the presence of the analyte molecule into a DNA signal. Detection systems associated with the substrate as provided herein are configured to generate signals that are attributable to individual binding sites.

### Sample

In some embodiments, the sample comprises an aqueous solution comprising protein, peptides, peptide fragments, lipids, DNA, RNA, organic molecules, inorganic molecules, complexes thereof, or any combinations thereof. In some embodiments, the analyte molecules in the sample comprise protein, peptides, peptide fragments, lipids, DNA, RNA, organic molecules, inorganic molecules, complexes thereof, or any combinations thereof. In some embodiments, the analyte molecules comprise comprises intact proteins, denatured proteins, partially or fully degraded proteins, peptide fragments, denatured nucleic acids, degraded nucleic acid fragments, complexes thereof, or combinations thereof. In some embodiments, the sample is obtained from tissue, cells, the environment of tissues and/or cells, or combinations thereof. In some embodiments, the sample comprises tissue biopsy, blood, blood plasma, urine, saliva, a tear, cerebrospinal fluid, extracellular fluid, cultures cells, culture media, discarded tissue, plant matter, synthetic proteins, bacterial, viral samples, fungal tissue, or combinations thereof. In some embodiments, the sample is isolated from a primary source such as cells, tissue, bodily fluids (e.g., blood), environmental samples, or combinations thereof, with or without purification. In some embodiments, the cells are lysed using a mechanical process or other cell lysis methods (e.g., lysis buffer). In some embodiments, the sample is filtered using a mechanical process (e.g., centrifugation), micron filtration, chromatography columns, other filtration methods, or combinations thereof. In some embodiments, the sample is treated with one or more enzymes to remove one or more nucleic acids or one or more proteins. In some embodiments, the sample comprises intact proteins, denatured proteins, partially or fully degraded proteins, peptide fragments, denatured nucleic acids or degraded nucleic acid fragments. In some embodiments, the sample is collected from one or more individual persons, one or more animals, one or more plants, or combinations thereof. In some embodiments, the sample is collected from an individual person, animal and/or plant having a disease or disorder that comprises an infectious disease, an immune disorder, a cancer, a genetic disease, a degenerative disease, a lifestyle disease, an injury, a rare disease, an age-related disease, or combinations thereof.

### Supramolecular Structure

In some embodiments, the supramolecular structure is a programmable structure that can spatially organize molecules. In some embodiments, the supramolecular structure comprises a plurality of molecules linked together. In some embodiments, the plurality of molecules of the supramolecular structure interact with at least some of each other. In some embodiments, the supramolecular structure comprises a specific shape. In some embodiments, the supramolecular nanostructure comprises a prescribed molecular weight based on the plurality of molecules of the supramolecular structure. In some embodiments, the supramolecular structure is a nanostructure. In some embodiments the plurality of molecules are linked together through a bond, a chemical bond, a physical attachment, or combinations thereof. In some embodiments, the supramolecular structure comprises a large molecular entity, of specific shape and molecular weight, formed from a well-defined number of smaller molecules interacting specifically with each other. In some embodiments, the structural, chemical, and physical properties of the supramolecular structure are explicitly designed. In some embodiments, the supramolecular structure comprises a plurality of subcomponents that are spaced apart according to a prescribed distance. In some embodiments, at least a portion of the supramolecular structure is rigid. In some embodiments, at least a portion of the supramolecular structure is semi-rigid. In some embodiments, at least a portion of the supramolecular structure is flexible.

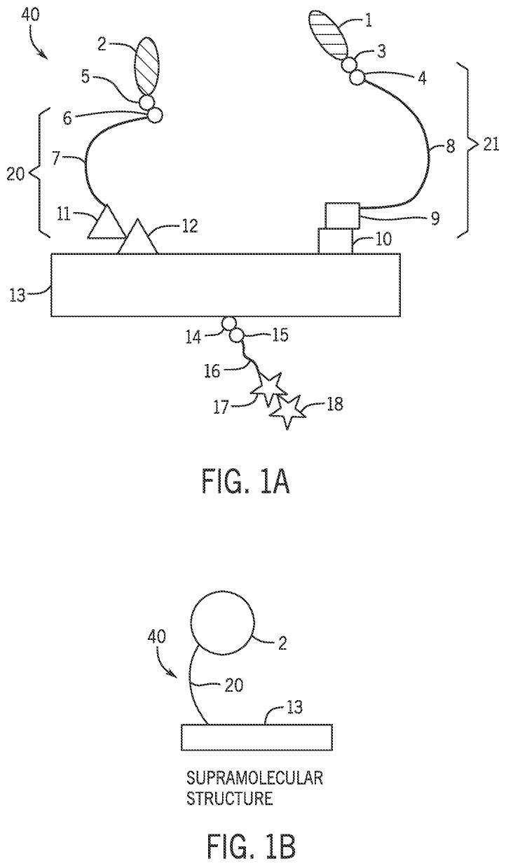

FIGS. 1A and 1B provide exemplary embodiments of a supramolecular structure 40 comprising a core structure 13 and a capture molecule 2. As shown in FIG. 1A, the supramolecular structure 40 can include, in embodiments, a detector molecule 1, and an anchor molecule 18. In some embodiments, the supramolecular structure comprises one or more capture molecules 2, and one or more detector molecules 1 and optionally one or more anchor molecules 18. In some embodiments, the supramolecular structure does not comprise an anchor molecule. In some embodiments, the supramolecular structure is a polynucleotide structure.

In some embodiments, the core structure 13 comprises one or more core molecules linked together. In some embodiments, the one or more core molecules comprise 2, 3, 4, 5, 6, 7, 8, 9, 10, 20, 50, 100, 200 or 500 unique molecules that are linked together. In some embodiments, the one or more core molecules comprises from about 2 unique molecules to about 1000 unique molecules. In some embodiments, the one or more core molecules interact with each other and define the specific shape of the supramolecular structure. In some embodiments, the plurality of core molecules interact with each other through reversible non-covalent interactions. In some embodiments, the specific shape of the core structure is a three-dimensional (3D) configuration. In some embodiments, the one or more core molecules provide a specific molecular weight. In some embodiments, the core structure 13 is a nanostructure. In some cases, the one or more core molecules comprise one or more nucleic acid strands (e.g., DNA, RNA, unnatural nucleic acids), one or more branched nucleic acids, one or more peptides, one or more small molecules, or combinations thereof. In some embodiments, the core structure comprises a polynucleotide structure. In some embodiments, at least a portion of the core structure is rigid. In some embodiments, at least a portion of the core structure is semi-rigid. In some embodiments, at least a portion of the core structure is flexible. In some embodiments, the core structure comprises a scaffolded deoxyribonucleic acid (DNA) origami, a scaffolded ribonucleic acid (RNA) origami, a scaffolded hybrid DNA/RNA origami, a single-stranded DNA tile structure, a multi-stranded DNA tile structure, a single-stranded DNA origami, a single-stranded RNA origami, a single-stranded RNA tile structure, a multi-stranded RNA tile structures, a hierarchically composed DNA and/or RNA origami with multiple scaffolds, a peptide structure, or combinations thereof. In some embodiments, the DNA origami is scaffolded. In some embodiments, the RNA origami is scaffolded. In some embodiments, the hybrid DNA/RNA origami is scaffolded. In some embodiments, the core structure comprising a DNA origami, RNA origami, or hybrid DNA/RNA origami that comprises a prescribed two-dimensional (2D) or 3D shape.

As shown in FIG. 1, in some embodiments, the core structure 13 is configured to be linked to a capture molecule 2, a detector molecule 1, an anchor molecule 18, or combinations thereof. In some embodiments, the capture molecule 2, detector molecule 1, and/or anchor molecule 18 are immobilized with respect to the core nanostructure 13 when linked thereto. In some embodiments, any number of the one or more core molecules comprises one or more core linkers 10,12,14 configured to form a linkage with a capture molecule 2, a detector molecule 1, and/or an anchor molecule 18. In some embodiments, any number of the one or more core molecules are configured to be linked with one or more core linkers 10,12,14 that are configured to form a linkage with a capture molecule 2, a detector molecule 1, and/or an anchor molecule 18. In some embodiments, one or more core linkers are linked to one or more core molecules through a chemical bond. In some embodiments, at least one of the one or more core linkers comprises a core reactive molecule. In some embodiments, each core reactive molecule independently comprises an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, at least one of the one or more core linkers comprises a DNA sequence domain.

In some embodiments, the core structure 13 is linked to 1) a capture molecule 2 at a prescribed first location on the core structure, 2) a detector molecule 1 at a prescribed second location on the core structure, and optionally 3) an anchor molecule 18 at a prescribed third location on the core structure. In some embodiments, a specified first core linker 12 is disposed at the first location on the core structure, and a specified second core linker 10 is disposed at the second location on the core structure. In some embodiments, one or more core molecules at the first location are modified to form a linkage with the first core linker 12. In some embodiments, the first core linker 12 is an extension of the core structure 13. In some embodiments, one or more core molecules at the second location is modified to form a linkage with the second core linker 10. In some embodiments, the second core linker 10 is an extension of the core structure 13. In some embodiments, the 3D shape of the core structure 13 and relative distances of the first and second locations are specified to maximize the intramolecular interactions between the capture molecule 2 and detector molecule 1. In some embodiments, the 3D shape of the core structure 13 and relative distances of the first and second locations are specified to obtain a desired distance between the capture molecule 2 and detector molecule 1, so as to maximize the intramolecular interactions between the capture molecule 2 and detector molecule 1.

As described herein, in some embodiments, the distance between the capture molecule 2 and detector molecule 1 is about 3 nm, 4 nm, 5 nm, 6 nm, 10 nm, 12 nm, 15 nm, 20 nm, 30 nm, or 40 nm. In some embodiments, the distance between the capture molecule 2 and detector molecule 1 is about 1 nm to about 60 nm. In some embodiments, the distance between the capture molecule 2 and detector molecule 1 is about 1 nm to about 2 nm, about 1 nm to about 5 nm, about 1 nm to about 10 nm, about 1 nm to about 20 nm, about 1 nm to about 40 nm, about 1 nm to about 60 nm, about 2 nm to about 5 nm, about 2 nm to about 10 nm, about 2 nm to about 20 nm, about 2 nm to about 40 nm, about 2 nm to about 60 nm, about 5 nm to about 10 nm, about 5 nm to about 20 nm, about 5 nm to about 40 nm, about 5 nm to about 60 nm, about 10 nm to about 20 nm, about 10 nm to about 40 nm, about 10 nm to about 60 nm, about 20 nm to about 40 nm, about 20 nm to about 60 nm, or about 40 nm to about 60 nm, including increments therein. In some embodiments, the distance between the capture molecule 2 and detector molecule 1 is about 1 nm, about 2 nm, about 5 nm, about 10 nm, about 20 nm, about 40 nm, or about 60 nm. In some embodiments, the distance between the capture molecule 2 and detector molecule 1 is at least about 1 nm, about 2 nm, about 5 nm, about 10 nm, about 20 nm, or about 40 nm. In some embodiments, the distance between the capture molecule 2 and detector molecule 1 is at most about 2 nm, about 5 nm, about 10 nm, about 20 nm, about 40 nm, or about 60 nm.

In some embodiments, a specified third core linker 14 is disposed at the third location on the core structure 13. In some embodiments, one or more core molecules at the third location is modified to form a linkage with the third core linker 14. In some embodiments, the third core linker 12 is an extension of the core structure 13. In some embodiments, the first and second locations are disposed on a first side of the core structure 13, and the optional third location is disposed on a second side of the core structure 13.

In some embodiments, the capture molecule 2 comprises a protein, a peptide, an antibody, an aptamers (RNA and DNA), a fluorophore, a nanobody, a darpin, a catalyst, a polymerization initiator, a polymer like PEG, an organic molecule, or combinations thereof. In some embodiments, the detector molecule 1 comprises a protein, a peptide, an antibody, an aptamers (RNA and DNA), a fluorophore, a nanobody, a darpin, a catalyst, a polymerization initiator, a polymer like PEG, an organic molecule, or combinations thereof. In some embodiments, the anchor molecule comprises a reactive molecule. In some embodiments, the anchor molecule 18 comprises a reactive molecule. In some embodiments, the anchor molecule 18 comprises a DNA strand comprising a reactive molecule. In some embodiments, the anchor molecule 18 comprises an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the anchor molecule 18 comprises a protein, a peptide, an antibody, an aptamers (RNA and DNA), a flourophore, a nanobody, a darpin, a catalyst, a polymerization initiator, a polymer like PEG, an organic molecule or combinations thereof. In some embodiments, a single pair of a capture molecule 2 and corresponding detector molecule 1 is linked to the core structure 13. In some embodiments, a plurality of pairs of capture molecules 2 and corresponding detector molecules 1 are linked to a core structure 13. In some embodiments, the plurality of pairs of capture molecules 2 and corresponding detector molecules 1 are spaced apart from each other to minimize cross-talk, i.e. minimizing capture and/or detector molecules from a first pair interacting with capture and/or detector molecules from a second pair.

In some embodiments, each component of the supramolecular structure may be independently modified or tuned. In some embodiments, modifying one or more of the components of the supramolecular structure may modify the 2D and 3D geometry of the supramolecular structure itself. In some embodiments, modifying one or more of the components of the supramolecular structure may modify the 2D and 3D geometry of the core structure. In some embodiments, such capability for independently modifying the components of the supramolecular nanostructure enables precise control over the organization of one or more supramolecular structures on solid substrate (e.g., planar surfaces) and 3D volumes (e.g., within a well formed on a solid substrate).

### Capture Barcode

As shown in FIGS. 1A and 1B, in some embodiments, the capture molecule 2 is linked to the core structure 13 through a capture barcode 20. In some embodiments, the capture barcode 20 forms a linkage with the capture molecule 2, and the capture barcode 20 forms a linkage with the core structure 13. In some embodiments, the capture barcode 20 comprises a first capture linker 11, a second capture linker 6, and a capture bridge 7. In some embodiments, the first capture linker 11 comprises a reactive molecule. In some embodiments, the first capture linker 11 comprises a reactive molecule comprising an amine, a thiol, a DBCO, a NHS ester, a maleimide, an azide, an acrydite, a single stranded nucleic acid (e.g., RNA or DNA) of specific sequence, or a polymer (e.g., polyethylene glycol (PEG) or one or more polymerization initiators). In some embodiments, the first capture linker 11 comprises a DNA sequence domain. In some embodiments, the second capture linker 6 comprises a reactive molecule. In some embodiments, the second capture linker 6 comprises a reactive molecule comprising an amine, a thiol, a DBCO, a NHS ester, biotin, a maleimide, an azide, an acrydite, a single stranded nucleic acid (e.g., RNA or DNA) of specific sequence, or a polymer (e.g., polyethylene glycol (PEG) or one or more polymerization initiators). In some embodiments, the second capture linker comprises a DNA sequence domain. In some embodiments, the capture bridge 7 comprises a polymer. In some embodiments, the capture bridge 7 comprises a polymer that comprises a nucleic acid (e.g., DNA or RNA) of a specific sequence. In some embodiments, the capture bridge 7 comprises a polymer such as PEG. In some embodiments, the first capture linker 11 is attached to the capture bridge 7 at a first terminal end thereof, and the second capture linker 6 is attached to the capture bridge 7 at a second terminal end thereof. In some embodiments, the first capture linker 11 is attached to the capture bridge 7 via a chemical bond. In some embodiments, the second capture linker 6 is attached to the capture bridge 7 via a chemical bond. In some embodiments, the first capture linker 11 is attached to the capture bridge 7 via a physical attachment. In some embodiments, the second capture linker 6 is attached to the capture bridge 7 via a physical attachment.

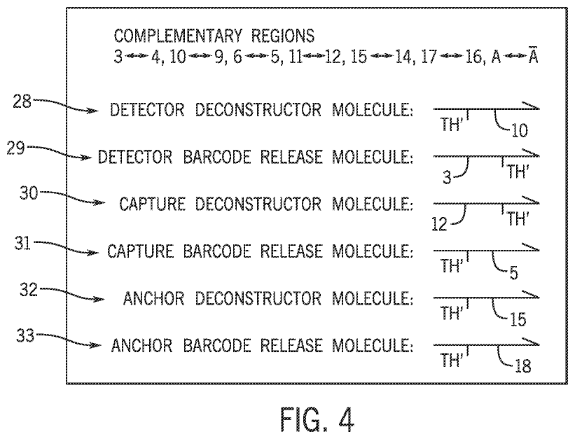

In some embodiments, the capture barcode 20 is linked to the core structure 13 through a linkage between the first capture linker 11 and the first core linker 12. In some embodiments, as described herein, the first core linker 12 is disposed at a first location on the core structure 13. In some embodiments, the first capture linker 11 and first core linker 12 are linked together through a chemical bond. In some embodiments, the first capture linker 11 and first core linker 12 are linked together through a covalent bond. In some embodiments, the linkage between the first capture linker 11 and first core linker 12 is reversible upon being subjected to a trigger. In some embodiments, the trigger comprises interaction with a deconstructor molecule (“capture deconstructor molecule”, e.g., reference character 30 in FIGS. 4,7) or exposure to a trigger signal. In some embodiments, the capture deconstructor molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, the capture barcode 20 is linked to the capture molecule 2 through a linkage between the second capture linker 6 and a third capture linker 5 that is bound to the capture molecule 2. In some embodiments, the third capture linker 5 comprises a reactive molecule. In some embodiments, the third capture linker 5 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the third capture linker 5 comprises a DNA sequence domain. In some embodiments, the capture molecule 2 is bound to the third capture linker 5 through a chemical bond. In some embodiments, the capture molecule 2 is bound to the third capture linker 5 through a covalent bond. In some embodiments, the second capture linker 6 and third capture linker 5 are linked together through a chemical bond. In some embodiments, the second linker 6 and third capture linker 5 are linked together through a covalent bond. In some embodiments, the linkage between the second capture linker 6 and third capture linker 5 is reversible upon being subjected to a trigger. In some embodiments, the trigger comprises interaction with a deconstructor molecule (“capture barcode release molecule”, e.g., reference character 31 in FIGS. 4,7) or exposure to a trigger signal. In some embodiments, the capture barcode release molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, being subject to a trigger breaks the linkage between the first capture linker 11 and first core linker 12 only, thereby breaking the capture molecule linkage with the core nanostructure 13 at the first location. In some embodiments, the capture barcode 20, when separated from the core structure 13 and the capture molecule 2, is configured to provide a signal for detecting an analyte molecule. In some embodiments, the signal as provided from the capture barcode 20 is a DNA signal.

### Detector Barcode

As shown in FIG. 1A, in some embodiments, the detector molecule 1 is linked to the core structure 13 through a detector barcode 21. In some embodiments, the detector barcode 21 forms a linkage with the detector molecule 1, and the detector barcode 21 forms a linkage with the core structure 13. In some embodiments, the detector barcode comprises a first detector linker 9, a second detector linker 4, and a detector bridge 8. In some embodiments, the first detector linker 9 comprises a reactive molecule. In some embodiments, the first detector linker 9 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the first detector linker 9 comprises a DNA sequence domain. In some embodiments, the second detector linker 4 comprises a reactive molecule. In some embodiments, the second detector linker 4 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the second detector linker 4 comprises a DNA sequence domain. In some embodiments, the detector bridge 8 comprises a polymer. In some embodiments, the detector bridge 8 comprises a polymer that comprises a nucleic acid (DNA or RNA) of a specific sequence. In some embodiments, the detector bridge 8 comprises a polymer such as PEG. In some embodiments, the first detector linker 9 is attached to the detector bridge 8 at a first terminal end thereof, and the second detector linker 4 is attached to the detector bridge 8 at a second terminal end thereof. In some embodiments, the first detector linker 9 is attached to the detector bridge 8 via a chemical bond. In some embodiments, the second detector linker 4 is attached to the detector bridge 8 via a chemical bond. In some embodiments, the first detector linker 9 is attached to the detector bridge 8 via a physical attachment. In some embodiments, the second detector linker 4 is attached to the detector bridge 8 via a physical attachment.

In some embodiments, the detector barcode 21 is linked to the core structure 13 through a linkage between the first detector linker 9 and the second core linker 10. In some embodiments, as described herein, the second core linker 10 is disposed at a second location on the core structure 13. In some embodiments, the first detector linker 9 and second core linker 10 are linked together through a chemical bond. In some embodiments, the first detector linker 9 and second core linker 10 are linked together through a covalent bond. In some embodiments, the linkage between the first detector linker 9 and second core linker 10 is reversible upon being subjected to a trigger. In some embodiments, the trigger comprises interaction with a deconstructor molecule (“detector deconstructor molecule”, e.g., reference character 28 in FIGS. 4,7) or exposure to a trigger signal. In some embodiments, the detector deconstructor molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, the detector barcode 21 is linked to the detector molecule 1 through a linkage between the second detector linker 4 and a third detector linker 3 bound to the detector molecule 1. In some embodiments, the third detector linker 3 comprises a reactive molecule. In some embodiments, the third detector linker 3 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the third detector linker 3 comprises a DNA sequence domain. In some embodiments, the detector molecule 1 is bound to the third detector linker 3 through a chemical bond. In some embodiments, the detector molecule 1 is bound to the third detector linker 3 through a covalent bond. In some embodiments, the second detector linker 4 and third detector linker 3 are linked together through a chemical bond. In some embodiments, the second detector linker 4 and third detector linker 3 are linked together through a covalent bond. In some embodiments, the linkage between the second detector linker 4 and third detector linker 3 is reversible upon being subjected to a trigger. In some embodiments, the trigger comprises interaction with a deconstructor molecule (“detector barcode release molecule”, e.g., reference character 29 in FIGS. 4,7) or exposure to a trigger signal. In some embodiments, the detector barcode release molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, being subject to a trigger breaks the linkage between the first detector linker 9 and second core linker 10 only, thereby breaking the detector molecule linkage with the core structure 13 at the second location. In some embodiments, the detector barcode 21, when separated from the core structure 13 and the detector molecule 2, is configured to provide a signal for detecting an analyte molecule. In some embodiments, the signal as provided from the detector barcode 21 is a DNA signal.

### Anchor Barcode

As shown in FIG. 1A, in some embodiments, the anchor molecule 18 is linked to the core structure 13 through an anchor barcode. In some embodiments, the anchor barcode forms a linkage with the anchor molecule 18, and the anchor barcode forms a linkage with the core structure 13. In some embodiments, the anchor barcode comprises a first anchor linker 15, a second anchor linker 17, and an anchor bridge 16. In some embodiments, the first anchor linker 15 comprises a reactive molecule. In some embodiments, the first anchor linker 15 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the first anchor linker 15 comprises a DNA sequence domain. In some embodiments, the second anchor linker 17 comprises a reactive molecule. In some embodiments, the second anchor linker 17 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the second anchor linker 17 comprises a DNA sequence domain. In some embodiments, the anchor bridge 16 comprises a polymer. In some embodiments, the anchor bridge 16 comprises a polymer that comprises a nucleic acid (DNA or RNA) of a specific sequence. In some embodiments, the anchor bridge 16 comprises a polymer such as PEG. In some embodiments, the first anchor linker 15 is attached to the anchor bridge 16 at a first terminal end thereof, and the second anchor linker 17 is attached to the anchor bridge 16 at a second terminal end thereof. In some embodiments, the first anchor linker 15 is attached to the anchor bridge 16 via a chemical bond. In some embodiments, the second anchor linker 17 is attached to the anchor bridge 16 via a physical attachment. In some embodiments, the first anchor linker 15 is attached to the anchor bridge 16 via a chemical bond. In some embodiments, the second anchor linker 17 is attached to the anchor bridge 16 via a physical attachment.

In some embodiments, the anchor barcode is linked to the core structure 13 through a linkage between the first anchor linker 15 and the third core linker 14. In some embodiments, as described herein, the third core linker 14 is disposed at a third location on the core structure 13. In some embodiments, the first anchor linker 15 and third core linker 14 are linked together through a chemical bond. In some embodiments, the first anchor linker 15 and third core linker 14 are linked together through a covalent bond. In some embodiments, the linkage between the first anchor linker 15 and third core linker 14 is reversible upon being subjected to a trigger. In some embodiments, the trigger comprises interaction with a deconstructor molecule (“anchor deconstructor molecule”, e.g., reference character 32 in FIGS. 4,7) or exposure to a trigger signal. In some embodiments, the anchor deconstructor molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, the anchor barcode is linked to the anchor molecule 18 through a linkage between the second anchor linker 17 and the anchor molecule 18. As disclosed herein, in some embodiments, the anchor molecule comprises a reactive molecule, a reactive molecule, a DNA sequence domain, a DNA sequence domain comprising a reactive molecule, or combinations thereof. In some embodiments, the anchor molecule 18 is bound to the second anchor linker 17 through a chemical bond. In some embodiments, the anchor molecule 18 is bound to the second anchor linker 17 through a covalent bond. In some embodiments, the linkage between the second anchor linker 17 and anchor molecule 18 is reversible upon being subjected to a trigger. In some embodiments, the trigger comprises interaction with a deconstructor molecule (“anchor barcode release molecule” e.g., reference character 33 in FIGS. 4,7) or exposure to a trigger signal. In some embodiments, the anchor barcode release molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, being subject to a trigger breaks the linkage between the first anchor linker 15 and third core linker 14 only, thereby breaking the anchor molecule linkage with the core structure 13 at the third location.

In some embodiments, the capture deconstructor molecule, capture barcode release molecule, detector deconstructor molecule, and detector barcode release molecule comprise the same type of molecule. In some embodiments, the capture deconstructor molecule, capture barcode release molecule, detector deconstructor molecule, and detector barcode release molecule comprise different types of molecules. In some embodiments, the capture deconstructor molecule, capture barcode release molecule, detector deconstructor molecule, detector barcode release molecule, anchor deconstructor molecule, and anchor barcode release molecule comprise the same type of molecules. In some embodiments, the capture deconstructor molecule, capture barcode release molecule, detector deconstructor molecule, detector barcode release molecule, anchor deconstructor molecule, and anchor barcode release molecule comprise different types of molecules. In some embodiments, any combination of the capture deconstructor molecule, capture barcode release molecule, detector deconstructor molecule, detector barcode release molecule, anchor deconstructor molecule, and anchor barcode release molecule comprise the same type of molecules.

### Three Arm Nucleic Acid Junction Based Supramolecular Structure

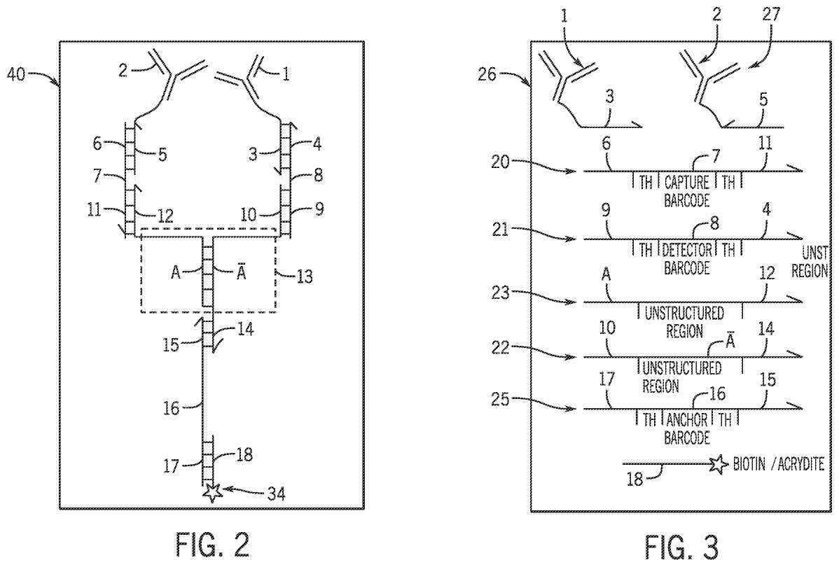

FIGS. 2-3 provides an exemplary depiction of a supramolecular structure 40 comprising a three arm nucleic acid junction and related subcomponents. FIG. 2 provides the complete supramolecular structure, while FIG. 3 provides the subcomponents that make up the supramolecular structure from FIG. 2. In some embodiments, the subcomponents of the supramolecular structure comprises five (5) DNA strands (ref characters 20-24), one (1) DNA strand with a terminal modification 25, and two (2) antibodies (1,2) modified with a single DNA linker 3,5. FIG. 4 provides an exemplary depiction of the respective deconstructor molecules configured to cleave a respective subcomponent from the supramolecular structure 40 in FIG. 2. The reference characters 1-18 in FIGS. 2-4 correspond to the respective components as provided with the same reference characters in FIG. 1A.

As shown in FIGS. 2-3, in some embodiments of a supramolecular structure, the core structure comprises two strands, a first core strand 23 and a second core strand 24 that each comprise partially complementary DNA sequence domains labelled A and A respectively in the FIGS. 2-4.

In some embodiments, the first core strand 23 of the core structure comprises a first core linker 12 comprising a DNA sequence domain. In some embodiments, the first core strand 23 comprises the DNA sequence domain labelled as “A” in FIGS. 2-4, which is separated from the first core linker 12 by an unstructured DNA region. In some embodiments, the unstructured DNA region comprises a polymer spacer. In some embodiments, the polymer spacer comprises a nucleic acid (DNA or RNA) of a specific sequence. In some embodiments, the polymer spacer comprises a polymer such as PEG.

In some embodiments, the first core linker 12 is complementary to a first capture linker 11 on the capture barcode strand 20. In some embodiments, the capture barcode strand 20 comprises a DNA strand comprising the first capture linker 11 and a second capture linker 6 at either end of said capture barcode strand 20. In some embodiments, the first capture linker 11 comprises a DNA sequence domain. In some embodiments, the second capture linker 6 comprises a DNA sequence domain. In some embodiments, the capture barcode strand 20 further comprises a unique capture barcode sequence 7 in between the first and second capture linkers 11, 6. In some embodiments, the unique capture barcode sequence 7 comprises a nucleic acid (DNA or RNA) of a specific sequence. In some embodiments, the unique capture barcode sequence 7 comprises a polymer such as PEG. In some embodiments, the capture barcode 20 comprises a short domain called the toeholds (“TH”). In some embodiments, the capture barcode sequence 7 comprises the toeholds (“TH”).

In some embodiments, the second capture linker 6 is complementary to a third capture linker 5. In some embodiments, the third capture linker 5 is a DNA sequence domain. In some embodiments, a capture molecule 2 is bound 27 to the third capture linker 5. In some embodiments, the capture molecule 2 is covalently bound to the third capture linker 5. In some embodiments, the capture molecule 2 is a capture antibody.

In some embodiments, the second core strand 24 of the core structure comprises a second core linker 10 comprising a DNA sequence domain. In some embodiments, the second core strand 24 comprises the DNA sequence domain labelled as “A” in FIGS. 2-4, which is separated from the second core linker 10 by an unstructured DNA region. In some embodiments, the unstructured DNA region comprises a polymer spacer. In some embodiments, the polymer spacer comprises a nucleic acid (DNA or RNA) of a specific sequence. In some embodiments, the polymer spacer comprises a polymer such as PEG. In some embodiments, the second core strand 24 further comprises a third core linker 14 adjacent to the sequence domain “A”. In some embodiments, the third core linker 14 comprises a DNA sequence domain.

In some embodiments, the second core linker 10 is complementary to a first detector linker 9 on the detector barcode strand 21. In some embodiments, the detector barcode strand 21 comprises a DNA strand comprising the first detector linker 9 and a second detector linker 4 at either end of the detector barcode section 21. In some embodiments, the first detector linker 9 comprises a DNA sequence domain. In some embodiments, the second detector linker 4 comprises a DNA sequence domain. In some embodiments, the detector barcode strand 21 further comprises a unique detector barcode sequence 8 in between the first and second detector linkers 9, 4. In some embodiments, the unique detector barcode sequence 8 comprises a nucleic acid (DNA or RNA) of a specific sequence. In some embodiments, the unique detector barcode sequence 8 comprises a polymer such as PEG. In some embodiments, the detector barcode 21 comprises a short domain called the toeholds (“TH”). In some embodiments, the detector barcode sequence 8 comprises the toeholds (“TH”).

In some embodiments, the second detector linker 4 is complementary to a third detector linker 3. In some embodiments, the third detector linker 3 is a DNA sequence domain. In some embodiments, a detector molecule 1 is bound 26 to the third detector linker 3. In some embodiments, the detector molecule 1 is covalently bound to the third capture linker 3. In some embodiments, the detector molecule 1 is a detector antibody.

In some embodiments, the third core linker 14 is complementary to a first anchor linker 15 on the anchor barcode strand 22. In some embodiments, the anchor barcode strand 22 comprises a DNA strand comprising the first anchor linker 15 and a second anchor linker 17 at either end of the anchor barcode section 22. In some embodiments, the first anchor linker 15 comprises a DNA sequence domain. In some embodiments, the second anchor linker 17 comprises a DNA sequence domain. In some embodiments, the anchor barcode strand 22 further comprises a unique anchor barcode sequence 16 in between the first and second anchor linkers 15, 17. In some embodiments, the unique anchor barcode sequence 16 comprises a nucleic acid (DNA or RNA) of a specific sequence. In some embodiments, the unique anchor barcode sequence 16 comprises a polymer such as PEG. In some embodiments, the anchor barcode 22 comprises a short domain called the toeholds (“TH”). In some embodiments, the anchor barcode sequence 16 comprises the toeholds (“TH”).

In some embodiments, the second anchor linker 17 is complementary to the anchor molecule 18. In some embodiments, the anchor molecule 18 comprises a DNA sequence domain. In some embodiments, the anchor molecule 18 is linked 25 to a terminal modification 34. In some embodiments, the terminal modification 34 comprises a reactive molecule. In some embodiments, the terminal modification 34 comprises a reactive molecule. In some embodiments, the terminal modification 34 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators).

FIG. 4 provides an exemplary embodiment of deconstructor molecules that may be used to trigger different reactions on the supramolecular structure 40. In some embodiments, a detector deconstructor molecule 28 comprises of a TH′ domain, whose sequence is complementary to the TH domain on the detector barcode 21 and the second core linker 10 (e.g., a DNA sequence domain) on the second core strand 24. In some embodiments, the detector deconstructor molecule 28 is configured to cleave the link between the detector barcode 21 and the core structure (e.g., the second core strand 24). In some embodiments, a detector barcode release molecule 29 comprises of a TH′ domain, whose sequence is complementary to the TH domain on the detector barcode 21 and the third detector linker 3 (e.g., a DNA sequence domain). In some embodiments, the detector barcode release molecule 28 is configured to cleave the link between the detector barcode 21 and the detector molecule 1.

In some embodiments, a capture deconstructor molecule 30 comprises a TH′ domain, whose sequence is complementary to the TH domain on the capture barcode 20 and the first core linker 12 (e.g., a DNA sequence domain) on the first core strand 23. In some embodiments, a capture deconstructor molecule 30 is configured to cleave the link between the capture barcode 20 and the core structure (e.g., the first core strand 23). In some embodiments, a capture barcode release molecule 31 comprises a TH′ domain, whose sequence is complementary to the TH domain on the capture barcode 20, and the third capture linker 5 (e.g., a DNA sequence domain). In some embodiments, the capture barcode release molecule 31 is configured to cleave the link between the capture barcode 20 and the capture molecule 2.

In some embodiments, an anchor deconstructor molecule 32 comprises a TH′ domain, whose sequence is complementary to the TH domain on the anchor barcode 22 and third core linker 14 (e.g., a DNA sequence domain) on the second core strand. In some embodiments, the anchor deconstructor molecule 32 is configured to cleave the link between the anchor barcode 22 and the core structure (e.g., the second core strand 24). In some embodiments, an anchor barcode release molecule 33 comprises a “TH′” domain, whose sequence is complementary to the “TH” domain on the anchor barcode 22 and the anchor molecule 18 (e.g., a DNA sequence domain). In some embodiments, the anchor barcode release molecule 33 is configured to cleave the link between the anchor barcode 22 and the anchor molecule 18.

In some embodiments, each of the different DNA domain sequences (reference character 1, 2, 3, 4, 5, 6, 7, 8, 9, 10, 11, 12, 13, 14, 15, A, A, TH, Capture Barcode 20, Detector Barcode 21 and Anchor barcode 22) independently comprise nucleic acid sequences from about 2 nucleotides to about 80 nucleotides.

### DNA Origami Based Supramolecular Structure

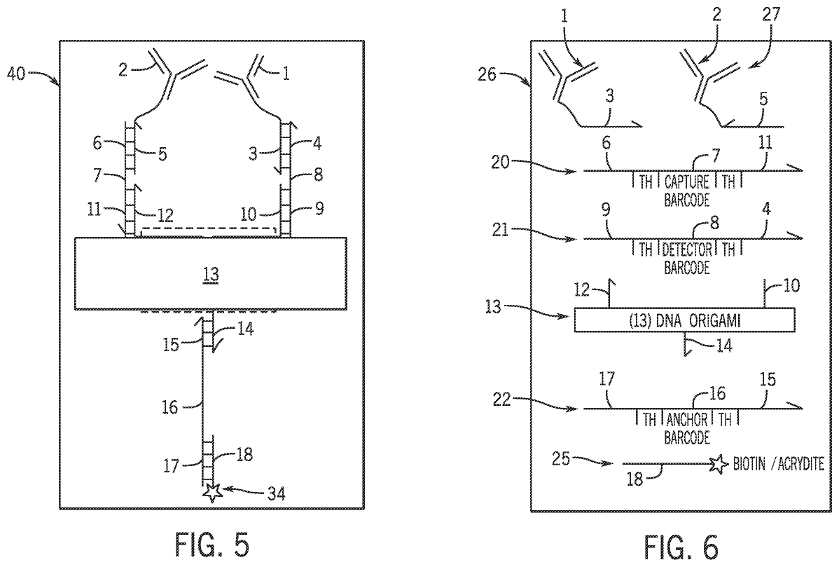

FIGS. 5-6 provides an exemplary depiction of a supramolecular structure 40 comprising a DNA origami and related subcomponents. FIG. 5 provides the complete supramolecular structure, while FIG. 6 provides the subcomponents that make up the supramolecular structure from FIG. 5. In some embodiments, the subcomponents of the supramolecular structure comprises a DNA origami 13 as a core structure, three (3) DNA strands (ref characters 20-22), one (1) DNA strand with a terminal modification 25, and two (2) antibodies (1,2) modified with a single DNA linker 3,5. FIG. 6 provides an exemplary depiction of the respective deconstructor molecules configured to cleave a respective subcomponent from the supramolecular structure 40 in FIG. 5. The reference characters 1-18 in FIGS. 5-7 correspond to the respective components as provided with the same reference characters in FIG. 1A.

In some embodiments, the core structure 13 comprises a scaffolded DNA origami, wherein a circular ssDNA molecule, called “scaffold” strand, is folded into a predefined 2D or 3D shape by interacting with 2 or more short ssDNA, called “staple” strands, which interact with specific sub-sections of the ssDNA “scaffold” strand.

As shown in FIGS. 5-6, in some embodiments of a supramolecular structure, the core structure 13 comprises a DNA origami. In some embodiments, the core structure 13 comprises a first core linker 12 comprising a DNA sequence domain. In some embodiments, the first core linker 12 is complementary to a first capture linker 11 on the capture barcode strand 20. In some embodiments, the capture barcode strand 20 comprises a DNA strand comprising the first capture linker 11 and a second capture linker 6 at either end of said capture barcode strand 20. In some embodiments, the first capture linker 11 comprises a DNA sequence domain. In some embodiments, the second capture linker 6 comprises a DNA sequence domain. In some embodiments, the capture barcode strand 20 further comprises a unique capture barcode sequence 7 in between the first and second capture linkers 11, 6. In some embodiments, the unique capture barcode sequence 7 comprises a nucleic acid (DNA or RNA) of a specific sequence. In some embodiments, the unique capture barcode sequence 7 comprises a polymer such as PEG. In some embodiments, the capture barcode 20 comprises a short domain called the toeholds (“TH”). In some embodiments, the capture barcode sequence 7 comprises the toeholds (“TH”).

In some embodiments, the second capture linker 6 is complementary to a third capture linker 5. In some embodiments, the third capture linker 5 is a DNA sequence domain. In some embodiments, a capture molecule 2 is bound 27 to the third capture linker 5. In some embodiments, the capture molecule 2 is covalently bound to the third capture linker 5. In some embodiments, the capture molecule 2 is a capture antibody.

In some embodiments, the core structure 13 comprises a second core linker 10 comprising a DNA sequence domain. In some embodiments, the second core linker 10 is complementary to a first detector linker 9 on the detector barcode strand 21. In some embodiments, the detector barcode strand 21 comprises a DNA strand comprising the first detector linker 9 and a second detector linker 4 at either end of the detector barcode section 21. In some embodiments, the first detector linker 9 comprises a DNA sequence domain. In some embodiments, the second detector linker 4 comprises a DNA sequence domain. In some embodiments, the detector barcode strand 21 further comprises a unique detector barcode sequence 8 in between the first and second detector linkers 9, 4. In some embodiments, the unique detector barcode sequence 8 comprises a nucleic acid (DNA or RNA) of a specific sequence. In some embodiments, the unique detector barcode sequence 8 comprises a polymer such as PEG. In some embodiments, the detector barcode 21 comprises a short domain called the toeholds (“TH”). In some embodiments, the unique detector barcode sequence 8 comprises the toeholds (“TH”).

In some embodiments, the second detector linker 4 is complementary to a third detector linker 3. In some embodiments, the third detector linker 3 is a DNA sequence domain. In some embodiments, a detector molecule 1 is bound 26 to the third detector linker 3. In some embodiments, the detector molecule 1 is covalently bound to the third capture linker 3. In some embodiments, the detector molecule 1 is a detector antibody.

In some embodiments, the core structure 13 comprises a third core linker 14 that comprises a DNA sequence domain. In some embodiments, the third core linker 14 is complementary to a first anchor linker 15 on the anchor barcode strand 22. In some embodiments, the anchor barcode strand 22 comprises a DNA strand comprising the first anchor linker 15 and a second anchor linker 17 at either end of the anchor barcode section 22. In some embodiments, the first anchor linker 15 comprises a DNA sequence domain. In some embodiments, the second anchor linker 17 comprises a DNA sequence domain. In some embodiments, the anchor barcode strand 22 further comprises a unique anchor barcode sequence 16 in between the first and second anchor linkers 15, 17. In some embodiments, the unique detector barcode sequence 16 comprises a nucleic acid (DNA or RNA) of a specific sequence. In some embodiments, the unique detector barcode sequence 16 comprises a polymer such as PEG. In some embodiments, the anchor barcode 22 comprises a short domain called the toeholds (“TH”). In some embodiments, the anchor barcode sequence 16 comprises the toeholds (“TH”).

In some embodiments, the second anchor linker 17 is complementary to the anchor molecule 18. In some embodiments, the anchor molecule 18 comprises a DNA sequence domain. In some embodiments, the anchor molecule 18 is linked 25 to a terminal modification 34. In some embodiments, the terminal modification 34 comprises a reactive molecule. In some embodiments, the terminal modification 34 comprises a reactive molecule comprising an amine, a thiol, a DBCO, a NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the supramolecular structure 40 does not include the anchor molecule 18 or associated core linker 14, anchor linker 15, barcode 16 or anchor linker 17, and the core structure 13 directly interacts with or contacts the substrate, as discussed herein.

FIG. 6 provides an exemplary embodiment of deconstructor molecules that may be used to trigger different reactions on the supramolecular structure 40. In some embodiments, a detector deconstructor molecule 28 comprises of a TH′ domain, whose sequence is complementary to the TH domain on the detector barcode 21 and the second core linker 10 (e.g., a DNA sequence domain) on the core nanostructure 13. In some embodiments, the detector deconstructor molecule 28 is configured to cleave the link between the detector barcode 21 and the core structure 13. In some embodiments, a detector barcode release molecule 29 comprises of a TH′ domain, whose sequence is complementary to the TH domain on the detector barcode 21 and the third detector linker 3 (e.g., a DNA sequence domain). In some embodiments, the detector barcode release molecule 28 is configured to cleave the link between the detector barcode 21 and the detector molecule 1.

In some embodiments, a capture deconstructor molecule 30 comprises a TH′ domain, whose sequence is complementary to the TH domain on the capture barcode 20 and the first core linker 12 (e.g., a DNA sequence domain) on the core nanostructure 13. In some embodiments, a capture deconstructor molecule 30 is configured to cleave the link between the capture barcode 20 and the core structure 13. In some embodiments, a capture barcode release molecule 31 comprises a TH′ domain, whose sequence is complementary to the TH domain on the capture barcode 20, and the third capture linker 5 (e.g., a DNA sequence domain). In some embodiments, the capture barcode release molecule 31 is configured to cleave the link between the capture barcode 20 and the capture molecule 2.

In some embodiments, an anchor deconstructor molecule 32 comprises a TH′ domain, whose sequence is complementary to the TH domain on the anchor barcode 22 and third core linker 14 (e.g., a DNA sequence domain) on the core nanostructure 13. In some embodiments, the anchor deconstructor molecule 32 is configured to cleave the link between the anchor barcode 22 and the core structure 13. In some embodiments, an anchor barcode release molecule 33 comprises a TH′ domain, whose sequence is complementary to the TH domain on the anchor barcode 22 and the anchor molecule 18 (e.g., a DNA sequence domain). In some embodiments, the anchor barcode release molecule 33 is configured to cleave the link between the anchor barcode 22 and the anchor molecule 18.

### Stable and Unstable State of Supramolecular Structure

In some embodiments, the supramolecular structure comprises one or more stable state configurations. In some embodiments, the supramolecular structure comprises one or more unstable state configurations. In some embodiments, the supramolecular structure comprises a bi-stable configuration having a stable state configuration and an unstable state configuration. In some embodiments, the two states, stable and unstable are defined based on the ability of an individual supramolecular structure to remain structurally intact when subjected to a unique molecule (e.g., a deconstructor molecule) and/or a trigger signal. In some embodiments, when the supramolecular structure is in the stable state, then all the different components that are part of the supramolecular structure remain physically connected to each other even after being exposed to the deconstructor molecule and/or trigger signal. In some embodiments, when the supramolecular structure is in the unstable state, then the exposure to the deconstructor molecule and/or trigger signal leads to a defined section (e.g., one or more subcomponents) of the supramolecular structure being physically cleaved, i.e. unbound (separated) from the supramolecular structure. In some embodiments, the supramolecular structure is configured to shift from a stable state to an unstable state upon interaction with an analyte molecule (as described herein). In some embodiments, the supramolecular structure is configured to shift from a unstable state to a stable state upon interaction with an analyte molecule (as described herein). In some embodiment, the analyte molecule that triggers the state change of the supramolecular structure comprises a protein, clusters of proteins, peptide fragments, cluster of peptide fragments, DNA, RNA, DNA nanostructure, RNA nanostructures, lipids, an organic molecule, an inorganic molecule, or any combination thereof.

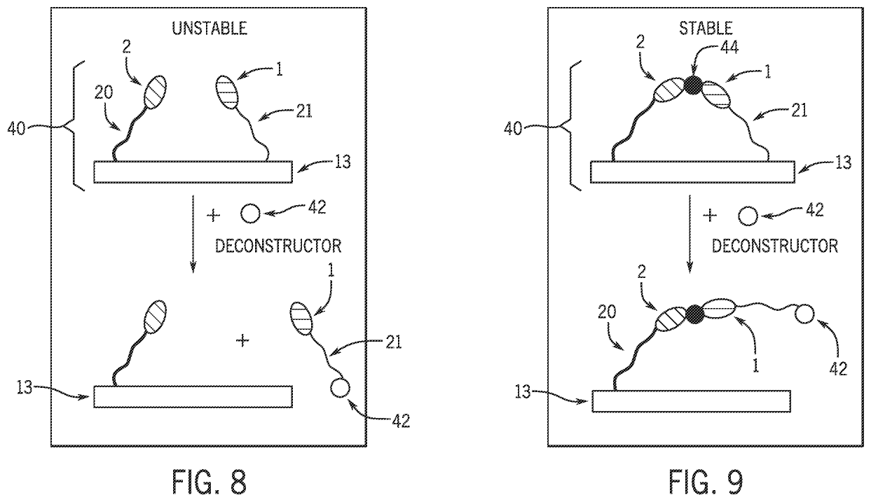

In some embodiments, a supramolecular structure in an unstable state configuration comprises a physical state wherein a linkage between the core structure 13 and a capture molecule 2 may be cleaved such that the capture molecule 2 is unbound from the core nanostructure 13. In some embodiments, the unstable state configuration comprises a physical state wherein a linkage between the core nanostructure 13 and a detector molecule 1 may be cleaved such that the detector molecule 1 is unbound from the core nanostructure 13. In some embodiments, the unstable state configuration comprises a physical state wherein a linkage between the core nanostructure 13 and a capture molecule 2 and a linkage between the core nanostructure 13 and a detector molecule 1 may be cleaved such that the capture molecule 2 and detector molecule 1 are unbound from the core nanostructure 13. In some embodiments, the linkage between the core nanostructure 13 and 1) the capture molecule 2, 2) the detector molecule 1, or 3) both, are cleaved upon being subjected to a trigger (e.g., a deconstructor molecule as described herein or trigger signal as described herein). FIG. 8 provides an exemplary depiction of a supramolecular structure 40 in an unstable state, wherein the detector molecule 1 is initially bound to the core structure 13 via a linkage with the detector barcode 21. With continued reference to FIG. 8, interaction with a deconstructor molecule 42 (e.g., detector deconstructor molecule 28) subsequently cleaves the linkage between the detector barcode 21 and core structure 13, such that the detector molecule 1 is unbound from the core nanostructure 13. In some embodiments, in the unstable state, the capture molecule 2 and detector molecule 1 on the core nanostructure 13 are freely diffusing with respect to each other, constrained only by the physical configuration of the core nanostructure 13.

In some embodiments, the stable state configuration comprises a physical state wherein the capture molecule 2 remains bound to the core nanostructure 13 upon cleavage of a linkage between the core structure 13 and the capture molecule 2. In some embodiments, the stable state configuration comprises a physical state wherein the detector molecule 1 remains bound to the core structure 13 upon cleavage of a linkage between the core nanostructure 13 and the detector molecule 1. In some embodiments, the stable state configuration comprises a physical state wherein the capture molecule 2 and detector molecule 1 are proximally positioned with respect to each other. In some embodiments, the detector molecule 1 and capture molecule 2 are proximally positioned with respect to each other with, or without, explicit bond formation between each other. In some embodiments, the detector 1 and capture 2 molecules are linked to each other. In some embodiments, the detector 1 and capture 2 molecules are linked to each other through a chemical bond. In some embodiments, the detector 1 and capture 2 molecules are linked together through a linkage with another molecule located between the capture and detector molecules (e.g., a sandwich formation). In some embodiments, the detector and capture molecules are linked together through linkage with an analyte molecule 44 from a sample (as described herein). FIG. 9 provides an exemplary depiction of a supramolecular structure 40 in a stable state, wherein the capture molecule 2 is linked to the detector molecule 1 through linkage with an analyte molecule 44. With continued reference to FIG. 9, interaction with a deconstructor molecule 42 cleaves the linkage between the detector molecule 1 and core structure 13, but the detector molecule 1 remains bound to the core nanostructure 13 through the linkage with the capture molecule 2. As described further herein, in some embodiments, a capture and/or detector molecule is configured to form a linkage with one or more specific types of analyte molecule from the sample. In some embodiments, interaction with the deconstructor molecule and/or trigger signal does not cleave the linkage between the capture and detector molecules.

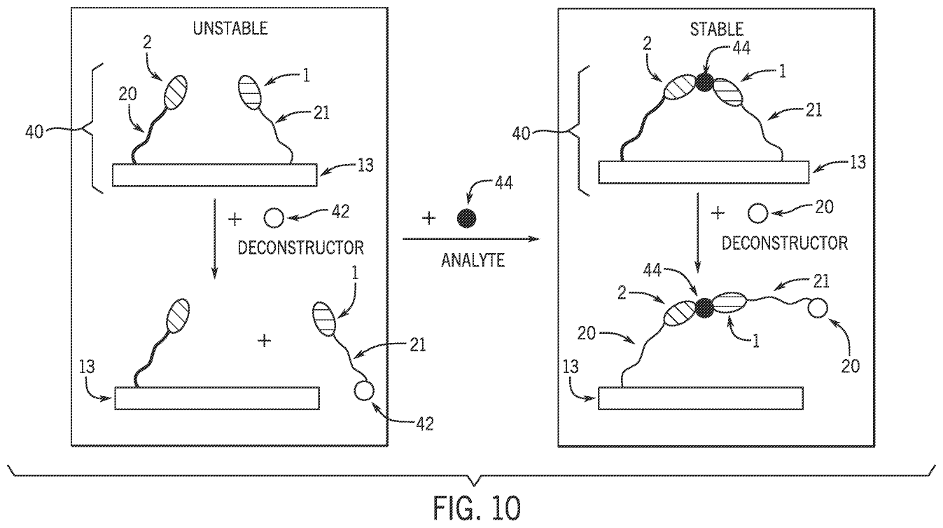

FIG. 10 provides an exemplary embodiment of a supramolecular structure shifting from an unstable state to a stable state. As described herein, a supramolecular structure 40 in an unstable state configuration will be separated from a detector molecule 1 (the detector molecule will be unbound from the supramolecular structure) upon interaction with a corresponding deconstructor molecule 42 (e.g., detector deconstructor molecule 28) and/or a trigger signal. With continued reference to FIG. 10, in some embodiments, interaction with an analyte molecule 44 from a sample binds the capture molecule and detector molecule together with the analyte molecule located therebetween (e.g., a sandwich formation), thereby shifting the supramolecular structure 40 from an unstable state to a stable state. In some embodiments, the analyte molecule 44 comprises a single molecule. In some embodiments, the analyte molecule instead comprises a plurality of analyte molecules. In some embodiments, the analyte molecule instead comprises a molecular cluster. In some embodiments, as described herein and shown in FIG. 10, with the supramolecular structure in a stable state, interaction with a corresponding deconstructor molecule cleaves the linkage between the core structure 13 and the detector barcode 21, wherein the detector molecule 1 remains linked to the core structure 13 through the linkage with the capture molecule 2 and analyte molecule 44.

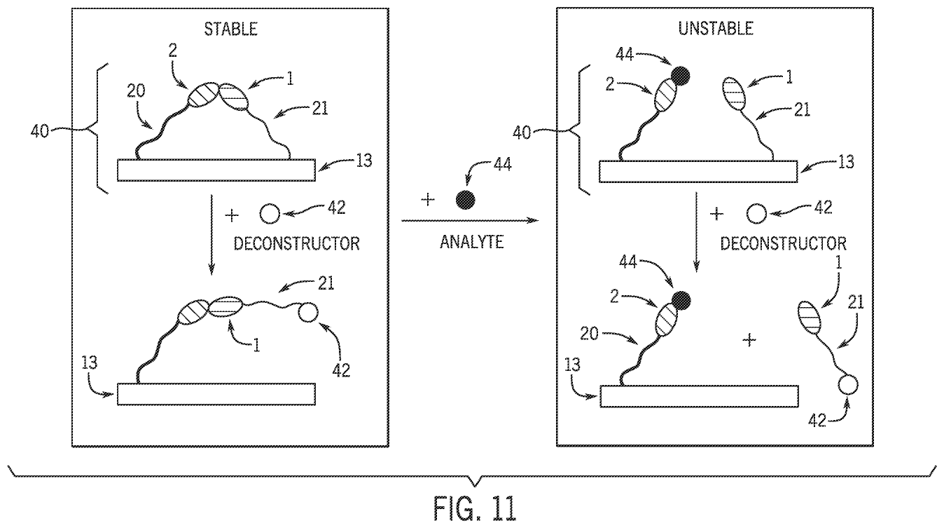

FIG. 11 provides an exemplary embodiment of a supramolecular structure 40 shifting from a stable state to an unstable state. As described herein, the supramolecular structure 40 is in a stable state configuration wherein the detector molecule 1 will remain linked to the core structure 13 upon interaction with a corresponding deconstructor molecule and/or trigger signal, due to the detector molecule 1 being linked to the capture molecule 2. With continued reference to FIG. 11, in some embodiments, interaction with an analyte molecule 44 from the sample cleaves the linkage between the capture molecule 2 and detector molecule 1, such that the analyte molecule 44 is bound to the capture molecule 1 only, thereby moving the supramolecular structure to an unstable state wherein the detector molecule 1 is bound to the core nanostructure 13 only through the linkage with the detector barcode 21. In some embodiments, the analyte molecule 44 comprises a single molecule. In some embodiments, the analyte molecule instead comprises a plurality of analyte molecules. In some embodiments, the analyte molecule instead comprises a molecular cluster. In some embodiments, as described herein and shown in FIG. 11, with the supramolecular structure in an unstable state, interaction with a corresponding deconstructor molecule 42 cleaves the linkage between the core structure 13 and the detector barcode 21, such that the detector molecule 1 is unbound (separated) from the core structure 13.

In some embodiments, the supramolecular structure 40 moves from a stable state to an unstable state upon interaction with an analyte molecule 44 that cleaves the linkage between a capture molecule 2 and detector molecule 1, wherein the analyte molecule 44 binds with the detector molecule 1. The capture molecule 2 is thereby unbound from the core structure 13 upon interaction with a corresponding destructor molecule 42 (e.g., capture deconstructor molecule 30).

### Methods for Detecting Analyte Molecules

As described herein, in some embodiments, one or more supramolecular structures enable the detection of one or more analyte molecules in a sample. In some embodiments, the supramolecular structure converts information about the presence of a given analyte molecule in a sample to a DNA signal. In some embodiments, the DNA signal corresponds to a capture barcode or detector barcode located on a supramolecular structure, wherein the capture molecule and detector molecule are simultaneously linked to the analyte molecule (e.g., sandwich formation). In some embodiments, capture and/or detector barcodes located on any unstable supramolecular structures are unbound therefrom using a trigger, such as a deconstructor molecule and/or a trigger signal. In some embodiments, the DNA signal is sequenced accordingly, and subsequently identified and correlated with the specific analyte molecule. Because, as provided herein, each supramolecular structure 40 may include at least one unique barcode, the location of an analyte binding event and transition of the supramolecular structure from the unstable state to the stable state may be linked to a particular binding site on the substrate by the barcode sequence.

In some embodiments, detecting the presence of an analyte molecule, as described herein, comprises controllably releasing a single, or multiple, unique nucleic acid molecules into the solution to be used to identify as well as quantify properties of the analyte molecule from the sample that triggered the state change of the supramolecular structure. In some embodiments, said unique nucleic acid molecules are provided by capture barcodes and/or detector barcodes of the respective supramolecular structures. In some embodiments, detecting the presence of an analyte molecule, as described herein, comprises creating an optical or electrical signal connected to the state change that can be counted to quantify the concentration of the analyte molecule in solution.

In some embodiments, a plurality of analyte molecules are simultaneously detected in a sample through multiplexing, wherein a plurality of supramolecular structures provide a plurality of signals (e.g., detector barcode, capture barcode) for sequencing and analyte identification. In some embodiments, methods described herein for detecting analytes in a sample provide a high-throughput and high-multiplexing capability by using a plurality of supramolecular structures. In some embodiments, the high-throughput and high-multiplexing capability provides high accuracy for analyte molecule detection and quantification. In some embodiments, methods described herein for detecting analytes in a sample are configured to characterize and/or identify biopolymers, including proteins molecules, quickly and at high sensitivity and reproducibility. In some embodiments, the plurality of supramolecular structures are configured to limit cross-reactivity associated errors. In some embodiments, such cross-reactivity associated errors comprise capture and/or detector molecules of a supramolecular structure interacting with capture and/or detector molecules of another supramolecular structure (e.g., intermolecular interactions). In some embodiments, each core structure of the plurality of supramolecular structures is identical to one another. In some embodiments, the structural, chemical, and physical property of each supramolecular structure is explicitly designed. In some embodiments, identical core structures have a prescribed shape, size, molecular weight, prescribed number of capture and detector molecules, predetermined distance between corresponding capture and detector molecules (as described herein), prescribed stoichiometry between corresponding capture and detector molecules, or combinations thereof, so as to limit the cross-reactivity between supramolecular structures. In some embodiments, the molecular weight of every core structure is identical and precise up to the purity of the core molecules. In some embodiments, each core structure has at least one capture molecule and at least one corresponding detector molecule.

In some embodiments, the plurality of supramolecular structures independently interact with different analyte molecules from a sample since the state change (from unstable to stable) is driven primarily by intramolecular interaction (capture and detector molecules on the same supramolecular structure). In some embodiments, the plurality of supramolecular structures might share structural similarities due to certain subcomponents being the same, however the interaction between an analyte molecule from the sample and supramolecular structure is defined by the corresponding capture molecule and detector molecule. In some embodiments, each pair of detector and capture molecules on a given supramolecular structure may specifically interact with a particular analyte molecule in the sample, leading to a state change of supramolecular structure upon interacting with the particular analyte molecule. In some embodiments, each supramolecular structure comprises unique DNA barcodes corresponding to the respective pair of detector and capture molecules. In some embodiments, a pair of detector and capture molecules on a given supramolecular structure is designed to interact with more than one analyte molecule in the sample.

In some embodiments, each supramolecular structure is configured for single-molecule sensitivity to ensure the highest possible dynamic range needed to quantitatively capture the wide range of molecular concentrations within a typical complex biological sample. In some embodiments, single-molecule sensitivity comprises the capture and detector molecules of a given supramolecular structure configured to shift from an unstable state to a stable state (or vice versa) through binding with a single analyte molecule. In some embodiments, the plurality of supramolecular structures limit or eliminate the manipulation of the sample needed to reduce non-specific interaction as well as any user induced errors.

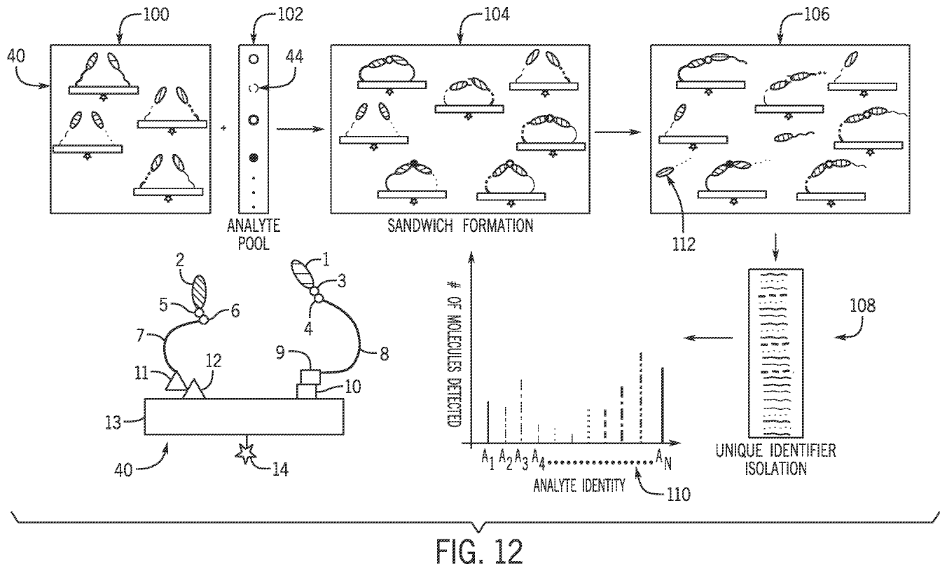

In some embodiments, the plurality of supramolecular structures are attached to one or more solid substrates, e.g., planar substrates or patterned substrates. FIG. 12 provides an exemplary method for detecting one or more analyte molecules in a sample using one or more supramolecular structures. In some embodiments, the sample, comprising one or more analytes (e.g., analyte pool 102) is contacted with the one or more supramolecular structures 40 (e.g., supramolecular structure pool 100). In some embodiments, as described herein, the plurality of supramolecular structures are provided as being attached to one or more solid substrates for single molecule organization as provided herein. In some embodiments, the sample is contact with the supramolecular structures for a time period from about 30 seconds to about 24 hours. In some embodiments, the sample is incubated with the supramolecular structures for a time period from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, from about 24 hours to about 48 hours.

With continued reference to FIG. 12, in some embodiments, the supramolecular structures are all in an unstable state (as shown with reference character 100). In some embodiments, and as described herein, interaction between an analyte molecule and corresponding capture 2 and detector 1 molecules shifts a respective supramolecular structure from an unstable state to a stable state (e.g., a sandwich formation with the capture molecule, analyte molecule, and detector molecule as shown with reference character 104). In some embodiments, a particular type of analyte molecule will bind with a particular pair of capture and detector molecules. In some embodiments, a given pair of capture and detector molecules are configured to bind with more than one type of analyte molecule. In some embodiments, the switching from an unstable state to a stable state for any given supramolecular structure is dependent on the specific capture and detector molecules bound thereto and the analyte molecules in the sample. In some embodiments, given that the state-change of the supramolecular structure is primarily dependent on the intra-molecular interactions (components located on the supramolecular structure), potential inter-molecular interactions between two different supramolecular structures are minimized or eliminated by limiting the net concentration of the supramolecular structures in the combined solution, such that the mean distance between any two supramolecular structures is larger than maximum intramolecular distance between a pair of capture and detector molecules on a given supramolecular structure.

As seen in FIG. 12, reference character 104, after contacting the sample, at least one of the supramolecular structures shifted to a stable state through interaction with an analyte molecule (e.g., sandwich formation wherein the capture molecule, analyte molecule and detector molecule are simultaneously linked together), while at least one of the supramolecular structures remained in an unstable state as the respective capture and detector molecules did not bind or interact with an analyte molecule from the sample.

After the sample has been contacted with the supramolecular structures for a prescribed amount of time, the combined solution of the sample and supramolecular structures, as shown in FIG. 12, is subjected to a trigger so as to cleave a linkage between the detector molecule and the core structure (reference character 106). In some embodiments, the trigger comprises introducing a solution comprising one or more deconstructor molecules (e.g., detector deconstructor molecule, reference character 28 from FIGS. 4,7) to the combined solution. In some embodiments, the trigger comprises subjecting the combined solution to a trigger signal. In some embodiments, the trigger comprises a combination of introducing a deconstructor molecule in the combined solution and subjecting the combined solution to a trigger signal. In some embodiments, as described herein, the deconstructor molecule comprises a nucleic acid (DNA or RNA), a peptide, a small organic molecule, or combinations thereof. In some embodiments, as described herein, the trigger signal comprises an electrical signal, microwave signal, ultraviolet illumination, visible illumination or near infra-red illumination. In some embodiments, the combined solution is subjected to the trigger for a prescribed amount of time. In some embodiments, the combined solution is incubated with one or more deconstructor molecules for a prescribed amount of time. In some embodiments, the combined solution is incubated with the deconstructor molecules for a time period from about 30 seconds to about 24 hours. In some embodiments, the combined solution is incubated with the deconstructor molecules for a time period from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, from about 24 hours to about 48 hours.

As shown in FIG. 12 reference character 106, in some embodiments, subjecting the combined solution to the trigger cleaves a linkage between the detector molecule and core structure of a supramolecular structure, such as the linkage between a detector barcode (e.g., reference character 21 from FIG. 1) and the core structure 13. In some embodiments, the cleavage is achieved through nucleic acid (DNA/RNA) strand displacement, optical cleavage, chemical cleavage, another technique known in the art, or combinations thereof. For supramolecular structures that shifted to a stable state, the detector molecule 1 is shown as remaining to be linked to the core structure 13 via linkage with the corresponding capture molecule 2. For supramolecular structures that remained in an unstable state, the detector molecule is shown as being unbound 112 from the respective supramolecular structure. In some embodiments, the unbound detector molecules 1 remain linked to the respective detector barcodes 21.

In some embodiments, the unbound detector molecules 1 (and corresponding detector barcode 21) are further separated from the combined solution. In some embodiments, the unbound detector molecules are separated from the combined solution through polyethylene glycol (PEG) precipitation. In some embodiments, the unbound detector molecules are separated from the combined solution by binding each core structure in the combined solution to a solid support through a corresponding anchor molecule on the respective core structure, followed by separation of the unbound detector molecules through centrifugation, micron filtration, chromatography or combinations thereof.

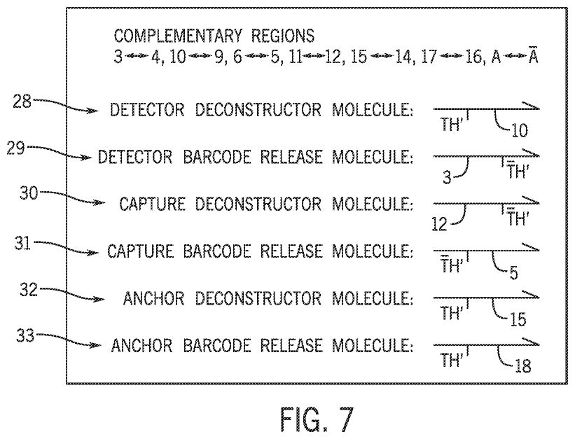

In some embodiments, after the unbound detector molecules have been separated from the combined solution, the detector barcodes 21 are cleaved from the corresponding detector molecules that are linked to a respective capture molecule (e.g., as located on a supramolecular structure that shifted to a stable state). In some embodiments, the detector barcodes 21 are cleaved from the corresponding detector molecules through nucleic acid (DNA/RNA) strand displacement, optical cleavage, chemical cleavage, or a combination thereof. In some embodiments, the detector barcodes are cleaved from the corresponding detector molecules by being subject to a trigger. In some embodiments, as described herein, the trigger comprises a deconstructor molecule, a trigger signal, or combinations thereof. In some embodiments, the deconstructor molecule comprises a detector barcode release molecule (e.g., reference character 29 from FIGS. 4 and 7).

In some embodiments, the cleaved detector barcodes 21 are isolated (reference character 108 FIG. 12) from the solution comprising the supramolecular structures. In some embodiments, the cleaved detector barcodes 21 are isolated from the solution through polyethylene glycol (PEG) precipitation. In some embodiments, the cleaved detector barcodes 21 are isolated from the solution by binding the core structures in the solution to a solid support through a corresponding anchor molecule on the respective core structure, followed by isolation of the cleaved detector barcodes through centrifugation, micron filtration, chromatography or combinations thereof.

In some embodiments, the cleaved detector barcodes provide a signal that correlates to the respective analyte molecule bound to the respective detector molecule. In some embodiments, as described herein, the detector barcode comprises a DNA strand. In some embodiments, the detector barcode provides a DNA signal correlating to the analyte molecule. In some embodiments, as depicted in FIG. 12 reference character 110, the isolated detector barcodes 21 are analyzed to identify and/or quantify the corresponding analyte molecules in the sample. In some embodiments the analysis of the isolated detector barcodes comprises genotyping, qPCR, sequencing, or combinations thereof.

In some embodiments, the method for detecting analyte molecules as depicted in FIG. 12 comprises cleaving the capture barcode 20 from a corresponding capture molecules that are linked to a respective detector molecule (e.g., as located on a supramolecular structure that shifted to a stable state). In some embodiments, the capture barcodes 20 are cleaved from the corresponding detector molecules through nucleic acid (DNA/RNA) strand displacement, optical cleavage, chemical cleavage, or a combination thereof. In some embodiments, the detector barcodes are cleaved from the corresponding detector molecules by being subject to a trigger. In some embodiments, as described herein, the trigger comprises a deconstructor molecule, a trigger signal, or combinations thereof. In some embodiments, the deconstructor molecule comprises a capture barcode release molecule (e.g., reference character 31 from FIGS. 4 and 7).

In some embodiments, the cleaved capture barcodes 20 are isolated (reference character 108 FIG. 12) from the solution comprising the supramolecular structures. In some embodiments, the cleaved capture barcodes 20 are isolated from the solution through polyethylene glycol (PEG) precipitation. In some embodiments, the cleaved capture barcodes 20 are isolated from the solution by binding the core structures in the solution to a solid support through a corresponding anchor molecule on the respective core structure, followed by isolation of the cleaved capture barcodes through centrifugation, micron filtration, chromatography or combinations thereof.

In some embodiments, the cleaved capture barcodes provide a signal that correlates to the respective analyte molecule bound to the respective detector molecule. In some embodiments, as described herein, the capture barcode comprises a DNA strand. In some embodiments, the capture barcode provides a DNA signal correlating to the analyte molecule. In some embodiments, as depicted in FIG. 12 reference character 110, the isolated capture barcodes 21 are analyzed to identify and/or quantify the corresponding analyte molecules in the sample. In some embodiments the analysis of the isolated capture barcodes comprises genotyping, qPCR, sequencing, or combinations thereof.

### Detection of Analyte Molecules Using a Surface Assay

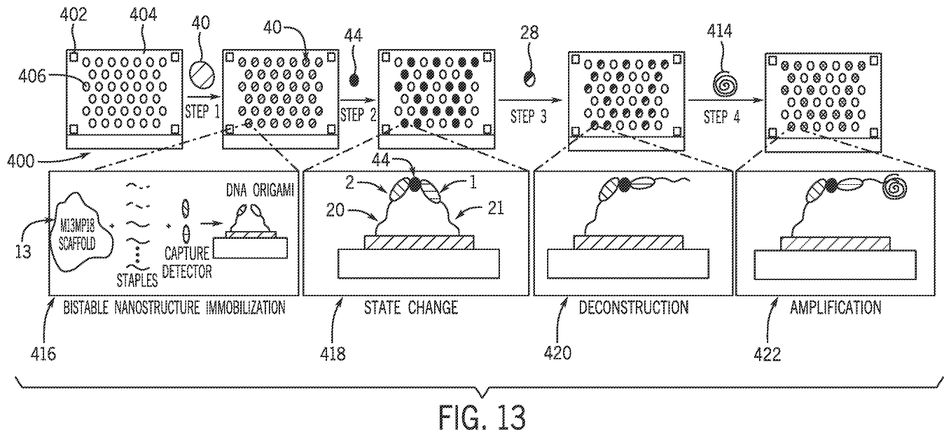

FIG. 13 provides an exemplary illustration of a technique for detecting analyte molecules in a sample using a surface based assay that uses supramolecular structures, as described herein, for single-molecule counting of analytes in the sample (i.e. detecting analyte molecules in the sample at a single molecule resolution). In some embodiments, the supramolecular structures comprise a core structure comprising a DNA origami core. In some embodiments, a planar substrate 400 is provided comprising (a) Fiduciary markers 402 that serve as reference coordinate for all the features on the substrate 400; (b) A defined set of micropatterned binding sites 406 where individual core structures (e.g., DNA origami) may be immobilized; (c) background passivation 404 that minimizes or prevents interaction between the surface of the substrate 400 and the supramolecular structure (including capture and detector molecules, core structure molecules) in areas that are outside of the binding sites 406. The substrate 400 may be a generally planar substrate, which should be understood to encompass substrates with micropatterned wells or protrusions on a surface. In some embodiments, the fiduciary markers comprise geometric features defined on a surface to be used as reference features for other features on the substrate. In some embodiments, the fiduciary markers 402 are coated with a polymer or self-assembled monolayer that does not interact with a core structure or other molecules of the supramolecular structure (e.g., DNA origami). In some embodiments, the background passivation 404 minimizes or prevents interaction between the surface of the substrate 400 and analyte molecules of the sample. In some embodiments, the planar substrate 400 comprises optical or electrical devices like FET, ring resonators, photonic crystals or microelectrode, to be defined prior to the formation of the binding sites 406. In some embodiments, the binding sites 406 are micropatterned on the planar substrate 400. In some embodiments, the binding sites 406 on the surface are in a periodic or regular pattern. In some embodiments, the binding sites 406 on the surface are in a non-periodic pattern (e.g., random). In some embodiments, a minimum distance (pitch) is specified between any two binding sites 406. In some embodiments, the minimum distance between any two binding sites 406 is at least about 200 nm. In some embodiments, the minimum distance between any two binding sites 406 is from at least about 40 nm to about 5000 nm. In some embodiments, the geometric shape of the binding sites 406 comprises a circle, square, triangle or other polygon shapes. In some embodiments, the chemical groups that are used for passivation 404 comprise neutrally charged molecules like a Tri-methyl silyl (TMS), an uncharged polymer like PEG a zwitterionic polymer like, or combinations thereof. In some embodiments, the chemical group used to define the binding site 406 comprises a silanol group, carboxyl group, thiol, other groups, or combinations thereof.

In some embodiments, a single supramolecular structure 40 is attached to a respective binding site 406 (Step 1). Reference character 416 provides a depiction of the components of the supramolecular structure 40, individually and as assembled and arranged on the planar substrate (components are as described herein, e.g., FIGS. 1, 2-3, 5-6). In some embodiments, the supramolecular structure 40 comprises a core structure 13 comprising a DNA origami, wherein the supramolecular structures 40 is attached onto each of the binding sites using DNA origami placement technique (step 1). In some embodiments, the supramolecular structure 40 is assembled prior to being attached to a respective binding site 406. In some embodiments, the DNA origami comprises a unique shape and dimension, so as to facilitate binding to a binding site using the DNA origami placement technique. In some embodiments, DNA origami placement comprises a directed self-assembly technique for organizing individual DNA origami (e.g., a core structure) on a surface (e.g., micropatterned surface). In some embodiments, alternatively to the DNA origami placement, a reactive group of the supramolecular nanostructure 40 is bound to a DNA origami that has been pre-organized on the binding site. In some embodiments, both of these methods for binding a supramolecular nanostructure to a corresponding binding site rely on the ability to organize one or more molecules on a micropatterned binding site using the DNA origami placement technique. In some embodiments, the planar substrate could be stored for a significant period after this step, in a clean environment.

With continued reference to FIG. 13, in some embodiments, a sample (as described herein) comprising analyte molecules is contacted with the planar substrate (step 2). In some embodiments, the sample is contacted with the planar substrate using a flow-cell. In some embodiments, the sample is incubated on the planar substrate with the supramolecular structures attached to the binding sites 406. In some embodiments, the incubation period may be from about 30 seconds to about 24 hours. In some embodiments, the incubation period may be from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, from about 24 hours to about 48 hours.

In some embodiments, the analyte molecules 44, in the sample, interact with the supramolecular structures 40 on the planar surface 400. In some embodiments, a single copy of a specific analyte molecule 44 binds simultaneously with both the capture and detector molecules, such that the particular supramolecular structure switches from an unstable state to a stable state 418 (as described herein, e.g., FIGS. 8-10). In some embodiments, a single copy of a particular analyte might interact simultaneously with the capture and detector molecules that are already bound to each other and switch the supramolecular structure from a stable state to an unstable state (as described herein, e.g., FIG. 11).

With continued reference to FIG. 13, in some embodiments, the planar substrate is then subjected to a trigger. In some embodiments, the trigger comprises a deconstructor molecule (e.g., detector deconstructor molecule 28 in FIG. 7). In some embodiments, the trigger comprises a trigger signal. In some embodiments, as described herein, the deconstructor molecule (e.g. detector deconstructor molecule 28) comprises a nucleic acid (DNA or RNA), a peptide, a small organic molecule, or combinations thereof. In some embodiments, as described herein, the trigger signal comprises an electrical signal, microwave signal, ultraviolet illumination, visible illumination or near infra-red illumination. In some embodiments, deconstructor molecules associated with the supramolecular structures attached to the planar substrate is allowed to interact with said supramolecular structures. In some embodiments, the deconstructor molecules are introduced into the flow-cell containing the planar substrate. In some embodiments, the deconstructor molecule is incubated with the supramolecular structures from about 30 seconds to about 24 hours (step 3). In some embodiments, the incubation period may be from about 30 seconds to about 1 minute, from about 1 minute to about 5 minutes, from about 5 minutes to about 30 minutes, from about 30 minutes to about 1 hr, from about 1 hr to about 5 hours, from about 5 hours to about 12 hours, from about 12 hours to about 24 hours, from about 24 hours to about 48 hours.

In some embodiments, interaction with the deconstructor molecule cleaves the detector molecules and detector barcodes of all the supramolecular structures in the unstable state, such that these detector molecules and detector barcodes will be physically cleaved from the planar substrate 400. In some embodiments, the physically cleaved detector molecules and detector barcodes are removed during one or more buffer washes at the end of the incubation step. In some embodiments, wherein supramolecular structures on the planar substrate had shifted to a stable state, due to the capture of single analyte molecules, the corresponding detector molecules and detector barcodes are still linked to the supramolecular structure 420, and thereby stably bound to the planar substrate due to the analyte mediated sandwich formed between the corresponding detector and capture molecules (i.e. linkage between the capture molecule, analyte molecule, and detector molecule).

With continued reference to FIG. 13, in some embodiments, the detector barcode at the location of supramolecular structure that shifted to a stable state is used as a binding site 422 for a signaling element 414 (step 4). In some embodiments, the signaling element comprises a fluorescent molecule or microbead, a fluorescent polymer, highly charged nanoparticles or polymer. In some embodiments, one or more signaling elements are allowed to interact with the supramolecular structures on the planar structure. In some embodiments, the signaling elements are introduced into the flow-cell containing the planar substrates. In some embodiments, the detector barcode is used as a polymerization initiator for growth of highly fluorescent polymer in a process such as rolling circle amplification or hybridization chain reaction.

In some embodiments, introduction of the signaling element 414 as described with step 4 leads to a surface in which every individual analyte capture event (i.e. linkage between the capture molecule, detector molecule, and analyte molecule) leads to a signaling element being present at the location of the respective analyte (as linked with the capture and detector molecules). In some embodiments, the signaling element is optically active and can be measured using a microscope or integrated optically sensor within the planar substrate 400. In some embodiments, the signaling element is electrically active and may be measured using an integrated electrical sensor. In some embodiments, the signaling element is magnetically active and may be measured using an integrated magnetic sensor. In some embodiments, each signal event is associated with the capture of the same type of analyte molecule (a single copy of the same type of analyte molecule), determined by the corresponding detector and capture molecule, thus counting the number of locations where the signaling element is present gives the quantification of the analyte molecule in the sample.

In some embodiments, the method for detecting an analyte as described in FIG. 13 uses a supramolecular core wherein the core structure is bound to a DNA origami already organized on the surface of the planar substrate through a respective anchor moiety of the core structure.

In some embodiments, the method for detecting an analyte as described in FIG. 13 enables the detection of a single type of analyte molecule. In some embodiments, the method for detecting an analyte as described in FIG. 13 enables detection of a plurality of types of analyte molecules (multiplexed analyte molecule detection). In some embodiments, each supramolecular structure is barcoded to uniquely identify the respective capture and detector molecules associated, thereby enabling the respective analyte molecule captured to be identified. In some embodiments, each supramolecular structure is barcoded using the respective anchor molecule.

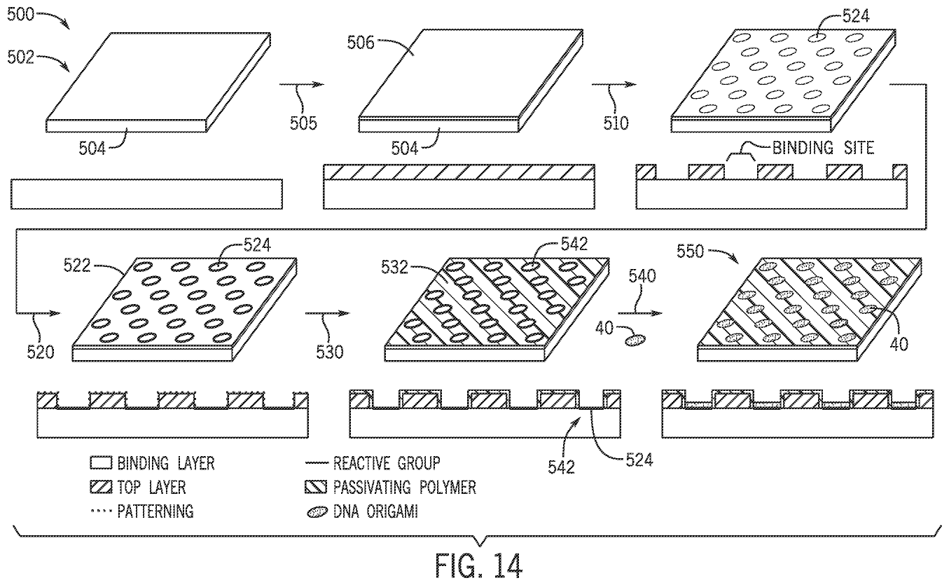

FIG. 14 is an example of a technique 500 for forming or manufacturing a substrate, such as the substrate 400 illustrated in FIG. 13. At step 502, a binding layer 504 is provided. The binding layer 504, in an embodiment, may be silicon, silicon dioxide, silicon nitride, graphene, quartz, metal, gold, silver, platinum, palladium, PDMS, polymer film, or combinations thereof. The binding layer 504 may be generally planar and may be cleaned prior to initiation of the method 500. At step 505, a top layer is deposited on the binding layer 504. The top layer 506, in an embodiment, may be graphene, aluminum oxide, HfO₂, Cr₂O₃ (Chromium oxide), Titanium oxide, Tantalum oxide, metal oxides, silicon dioxide (SiO₂) or combinations thereof. At step 510, the top layer 506 is patterned, e.g., by removing portions of the top layer 506, to expose locations 514 of the binding layer 504 that will correspond to binding sites of the substrate. The patterning may be photolithography, e-beam lithography, nanoimprinting, or other patterning modalities. The top layer 506 and/or the exposed region on the binding layer 504 may be activated by chemical or plasma treatment at step 520 to yield different reactive groups, depending on the individual chemistry of these layers. As illustrated, the top layer reactive groups 522 and binding layer reactive groups 524 may be generated via activation. The activation may be in a same or sequential steps.

A passivating layer 532, e.g., a passivating polymer is applied and only reacts with the top layer reactive groups 522 at step 530. The passivating polymer may be entropic, e.g., made from groups that uniquely interact with the top layer reactive groups 522 and not with the binding layer reactive groups 524. The top surface of the top layer 506 includes the passivating layer 532 and surrounds the binding sites 542. At step 540, supramolecular structures 40, such as DNA origami 540, are applied to the binding sites 542 and interact with the reactive groups 524 of the binding sites 542 such that each binding site 542 of the substrate 550 includes a supramolecular structure 40. It should be understood that, in embodiments, a substrate 550 may be considered to include a supramolecular structure 400 in each binding site 542 within a certain tolerance, e.g., if greater than 95% or greater than 97% of the binding sites 542 include at least one supramolecular structure 40. Further, certain binding sites 542 may be reserved for fiducials or control purposes. In some embodiments, each binding site 542 includes at most one or a single supramolecular structure 40. Binding sites 542 may have a defined shape and size created by the patterning technique of the top layer 506. The supramolecular structure 40 may be placed as a preformed unit or may be assembled in stages on the binding site 542. In one example, a core structure, e.g., a DNA origami portion, may first be associated with the binding site 542. Subsequent to the association, the capture molecule and the detector molecular may be linked to respective locations with the desired pre-determined spacing in the unstable state.

In some embodiments, the anchor molecule 18 (see FIG. 1), when present, forms the association with the binding layer reactive groups 524. However, it should be understood that the supramolecular structure 40 may not include the anchor molecule 18. The binding layer reactive groups 524 of the binding sites 542 may be configured to form salt bridges with nucleic acid molecules of the core structure to associate the supramolecular structure 40 with the binding site 542 via direct association of the core structure. That is, the reactive groups may be negatively charged and may react with negatively charged nucleic acid molecules, e.g., of a DNA origami, of the core structure to form a chemical association that resists removal during washing or other steps. The salt bridge association may be augmented with covalent links in embodiments.

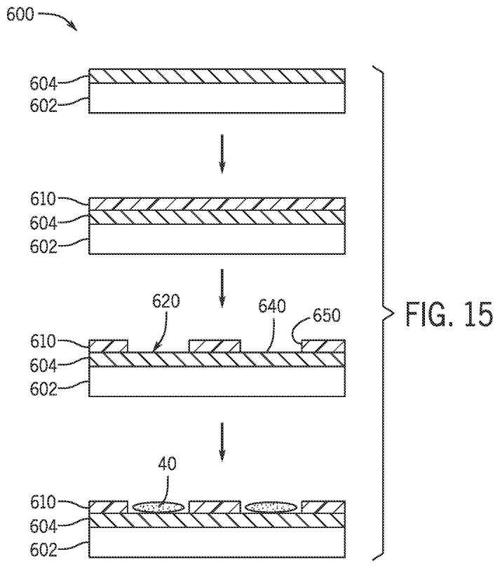

FIG. 15 is an example of a technique 600 for forming or manufacturing a substrate, such as the substrate 400 illustrated in FIG. 13. The technique 600 initiates with a starting base layer 602 and a binding layer 604. The base layer 602 may be a generally planar layer onto which the binding layer 604 is grown or deposited. In the illustrated embodiment, the base layer 602 is a silicon wafer and the binding layer 604 is a silicon dioxide that is grown on the surface of the base layer 602. While the illustrated example includes the binding layer 604 only one side of the base layer 602, the binding layer 604 may additionally or alternatively be present on the opposing surface of the base layer 602 to increase the reactive surface area of the substrate.

Subsequently, a thin film 610 of material (metal, metal oxide, polymer or any material desired to be patterned) is deposited onto the binding layer 604. In embodiments, an adhesion promoter may be applied to the top layer in order to enhance coupling of the thin film 610. The thickness of the thin film 610, in embodiments, could range from 2 Angstroms to 10 microns. The thickness of the thin film 610 will dictate the topography of the final patterned substrate. The thin film 610 may be patterned using conventional methods such as photolithography, nanoimprint lithography, direct writing methodologies, roll-to-roll embossing, etc. (lithography typically includes a pattern transfer process step that is not shown, this could include etching of the film). After etching of the thin film 610, the underlying surface 620 of the binding layer 604 is exposed. It is possible to have stacks of thin films 610 and etch through specific layers to expose different materials (chemistries). The underlying surface 620 may be subjected to additional procedures, such as activation, to generate the binding sites 640. Placement of the supramolecular structure 40, e.g., a DNA origami, onto the patterned surface then takes place.

The dimensions of the origami are designed a-priori to placement to define how many supramolecular structures 40 are placed at the capture site. The dimensions of each binding site 640 across the surface 620 may be considered in an x-y plane. In embodiments in which the binding site 640 is a well bounded by walls 650 of the thin film 610, the x-y dimensions are thus bounded by the walls 650. Thus, a physical space of the well may act as a barrier to more than one supramolecular structure 40 associating with the surface 620 of the binding site 640. In an embodiment, at least one dimension of the core structure of the supramolecular structure 40 is smaller than a dimension of the binding site 640 across the x-y plane to facilitate entry of the supramolecular structure 40 into the well. In an embodiment, at least one dimension of the core structure of the supramolecular structure 40 is at least 50% of a length of a dimension of the binding site across the x-y plane. Where the binding site 640 is a circle, the supramolecular structure 40, or the core structure, may have at least one dimension that is greater than a radius of the circle.

The chemistry of the thin film 610 may be modified before placement takes place through the specific growth of passivation films nucleating from the. Example modification can be through growth of SAMPs (self assembled monolayer phosphonates), silanization—deposited through conventional methods such as chemical vapor deposition). The surface chemistry (functional groups at the surface of the patterned wafer) is different between thin film 610 (or modified thin film 610) and the SiO₂ underling surface 620 exposed after patterning (or other thin film that may have been deposited before film thin film 610).

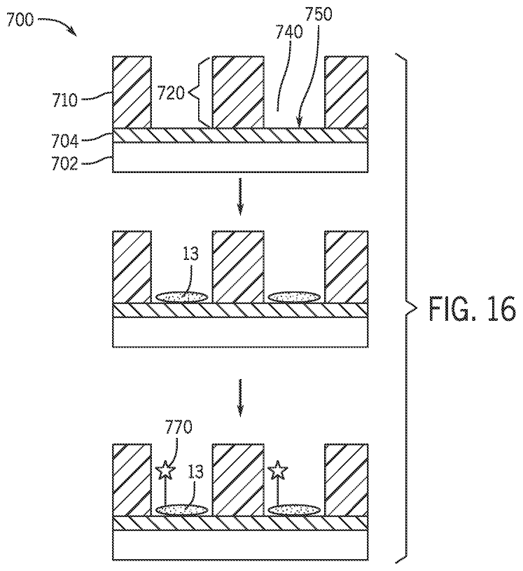

FIG. 16 is an example of a technique 700 for forming or manufacturing a substrate, such as the substrate 400 illustrated in FIG. 13, that includes a base layer 702, binding layer 704, and thin film 710. The binding sites may be formed as set forth in FIG. 15. Here, the thickness 720 of the thin film 710 is such that it is sufficiently larger than the DNA origami core structure 13 of supramolecular structure 40 is thick. By way of example, the thin film 610 is 100 nm thick and the origami of the core structure 13 is 1 nm thick. The core structure is placed into the wells of the binding sites 640 in a one-to-one correspondence. This can be accomplished through use of linking groups between the origami and complements deposited or activated specifically at the top surface 750 of the binding site 740 at the bottom of the well. Subsequent to loading the array with an excess of origami, linking of the origami can be performed and then a washing is performed to remove all but one origami from each well.

The thin film 710 can also be used for three-dimensional control of cargo elements 770. The binding site characteristics may include the x-y dimensions across the top surface 750 and a z dimension corresponding to the thickness 720 of the thin film 710.

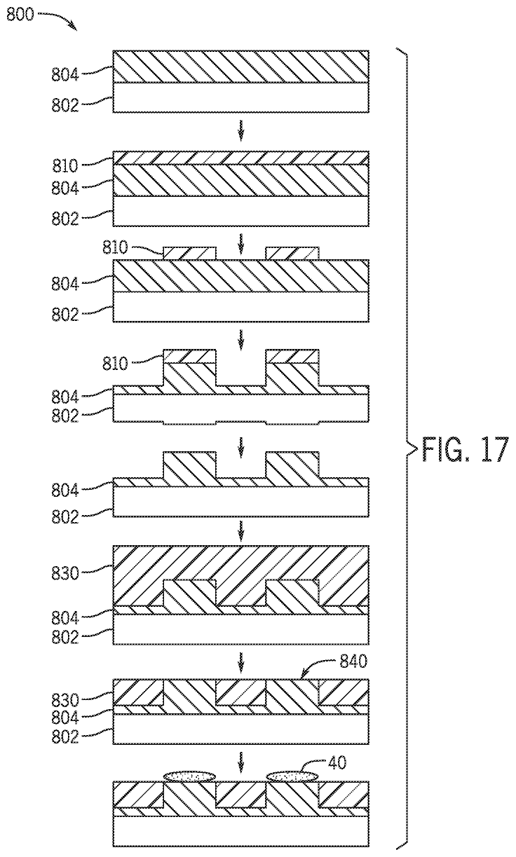

FIG. 17 is an example of a technique 800 for forming or manufacturing a substrate, such as the substrate 400 illustrated in FIG. 13. The technique 800 initiates with a starting base layer 802 and a binding layer 804. The base layer 802 may be a generally planar layer onto which the binding layer 804 is grown or deposited. In the illustrated embodiment, the base layer 802 is a planar support, such as a glass or silicon wafer, and the binding layer 804 is a silicon dioxide, silicon nitride, graphene or silicon carbide that is grown on the surface of the base layer 802. However, in certain embodiments, the base layer 802 is the same material as the binding layer 804 or the base layer 802 is not present. While the illustrated example includes the binding layer 804 only one side of the base layer 802, the binding layer 804 may additionally or alternatively be present on the opposing surface of the base layer 802 to increase the reactive surface area of the substrate.

Subsequently, the sacrificial film 810 is put onto the binding layer 804. This sacrificial film 810 could be a photoresist, a nanoimprint resist, a metal, or a similar material having the ability to be patterned. The sacrificial film 810 is patterned as provided herein into a desired pattern corresponding to a desired binding site pattern. That is, the sacrificial film 810 that remains after patterning corresponds to the eventual location of binding sites. The sacrificial film 810 is used as an etch mask to transfer the pattern into the binding layer 804. The pattern transfer process can be done with the use of reactive ion etching (plasma etching), solution phase etching. The depth of the etch can be controlled with exposure time of material to etchant or by hitting the underlying different material (Si shown here). The sacrificial film 810 will be subsequently removed, as shown. Subsequently, a conformally coated film 830 is deposited over the binding layer 804 and buries the etched or formed features created in the previous step. This can be done with sputtering, atomic layer deposition, electron beam evaporation, etc. The thickness of this new conformally coated film 830 is greater than the depth of the features etched in the previous step such that the features are not exposed until subsequent steps. The surface is planarized, e.g., through CMP (chemical mechanical polishing) or other suitable means to expose the topography defined in the previous steps. Placement of the supramolecular structures 40 onto the patterned surface then takes place. The dimensions of the supramolecular structures 40 are designed a-priori to placement to define how many origami are placed at the binding site 840. The chemistry of the conformally coated film 830 may be modified before placement takes place through the specific growth of passivation films nucleating from the surface. Example modification can be through growth of SAMPs (self assembled monolayer phosphonates), silanization—deposited through conventional methods such as chemical vapor deposition). The surface chemistry (functional groups at the surface of the patterned wafer) are different between conformally coated film 830 (or modified conformally coated film 830) and the binding layer 804 exposed after patterning (or other thin film that may have been deposited before conformally coated film 830).

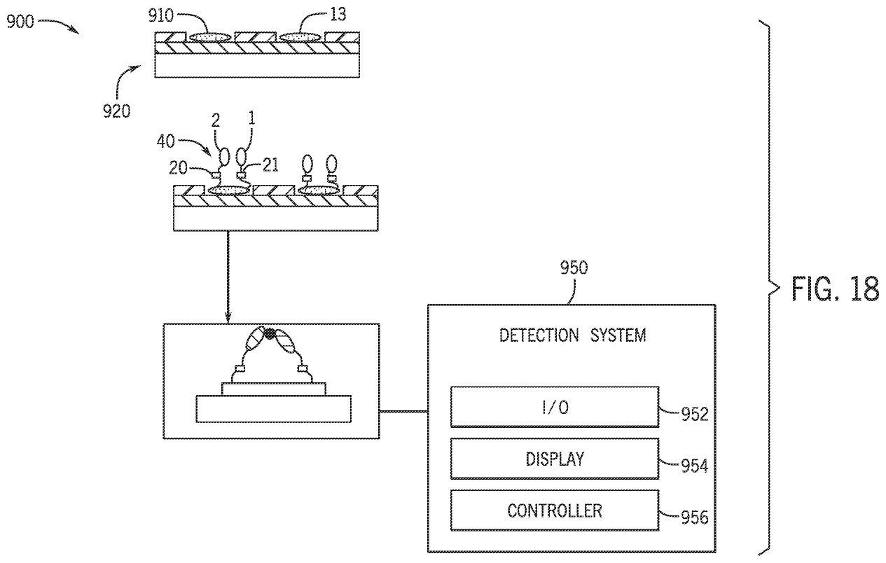

FIG. 18 shows an example of a supramolecular assembly process for analyte detection. In some embodiments, the core structure 13 (e.g., DNA origami) is placed at a binding site 910 of a patterned substrate 920 before being assembled as a supramolecular structure 40. Thus, each core structure 13, once in position at an individual binding site 910, can be customized to include desired analyte detection characteristics by linking to a capture molecule 2 at a prescribed first location on the core structure 13 and a detector molecule 1 at a prescribed second location on the core structure 13 and such that the linking is in the unstable configuration in the absence of analyte. Thus, the linking to capture/detection moieties may occur subsequent to placement of the core structure 13. Further, depending on the desired 3-dimensional characteristics various linkers subject to particular triggers and having particular lengths may be selected. As noted, these may be individualized per binding site, such that each binding site 910 on the patterned substrate 920 has known analyte detection characteristics that may be the same as or different than those in neighboring binding sites 910. In this manner, complexity and multiplexing of protein—protein or other detection modalities may be achieved.

In some embodiments, different capture barcodes 20 and detector barcodes 21 may be used to distinguish between different binding sites 910. Thus, the detection system 950 as provided herein may, in an embodiment, detect and characterize amplification of one or more barcode sequences as part of detection. In addition, each core structure 13 may be coupled to or include a barcode sequence.

Further, the detection system may additionally or alternatively be configured to detect electrical, optical, and/or magnetic environmental changes that are characteristic of analyte binding to the supramolecular structure 40. Such detection may be coordinated with amplification-based detection to generate information indicative of the presence and/or levels of the analyte as well as the identity of the capture and or detector molecules. Further, the capture and detector linking may be patterned or associated with known locations or binding sites.

The detection system 950 may include input/output circuitry 952, a display 954, and a processor-based controller 956 that executes instructions stored in a memory. The detection may, via the display 954, be configured to generate notifications or information related to analyte binding associated with a transition of the supramolecular structure to the stable state.

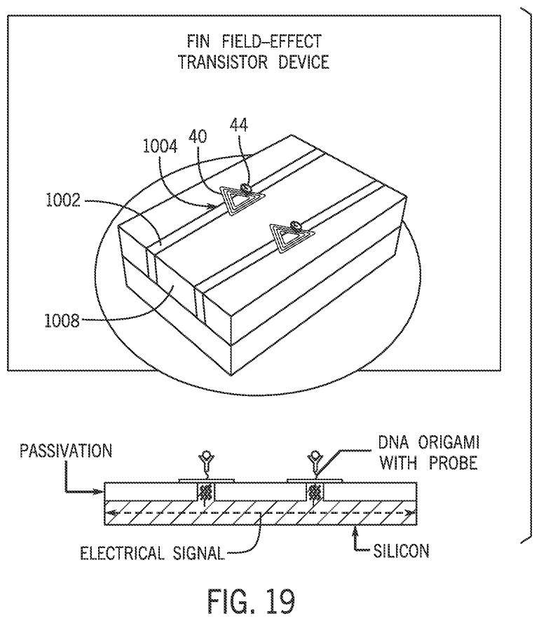

In some embodiments, the transition of the supramolecular structure 40 to the stable state as a result of binding of the analyte 44 may be detected via a field-effect transistor device, as shown in FIG. 19. Thus, the detection system 950 may be capable of detecting environmental changes characteristic of analyte binding 44 that are manifested as changes in detected electrical signals from one or more electrical leads 1002 coupled to each binding site 1004 and extending through a passivation layer 1008. In certain embodiments, each binding site 1004 is coupled to a dedicated lead 1002. In other embodiments, appropriate resolution may be achieved through multiple binding sites 1004 coupling to a single electrical lead 1002. Detected signals are provided to the detection device 950 for analysis.

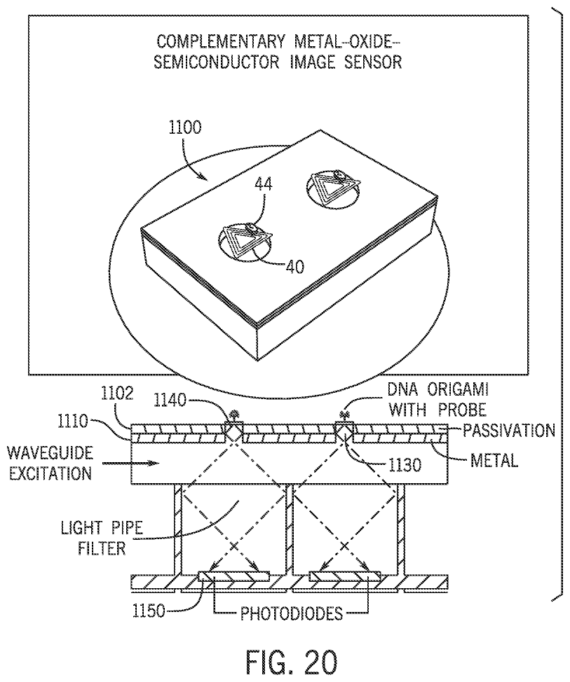

In some embodiments, the transition of the supramolecular structure 40 to the stable state as a result of binding of the analyte 44 may be detected via a metal-oxide semiconductor image sensor device, as shown in FIG. 20. Thus, the detection system 950 may be capable of detecting environmental changes characteristic of analyte binding 44 that are manifested as changes in detected optical signals. In the illustrated embodiment, the substrate 1100 includes a passivation layer 1102 and a metal oxide layer 1100. Light pipes 1130 extend through the passivation layer 1102 and metal oxide layer 1100 to couple to the binding sites 1140 and permit detection of optical changes characteristic of analyte binding by the photodetector 1150. Each binding site 1120 may be coupled to a dedicated light pipe 1130 and photodetector 1150. Detected signal are provided to the detection device 950 for analysis. It should be understood that the disclosed detection modalities are by way of example, and other implementations are also contemplated.

### Substrate Regeneration

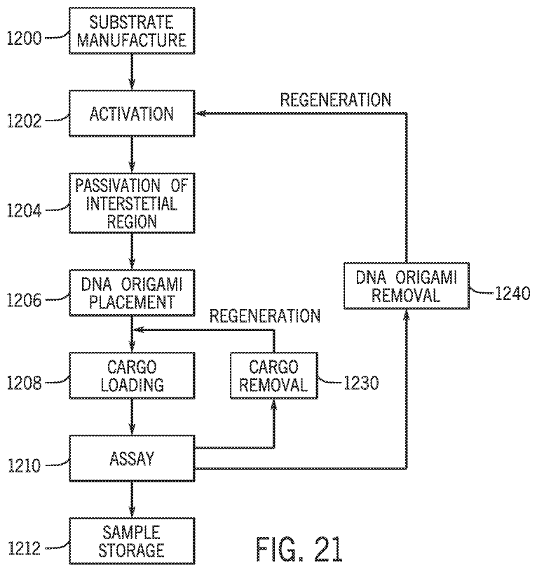

A feature of the disclosed techniques is that the surfaces of the organized substrates, once patterned and used for analyte detection, can be regenerated all or in part and reused to detect the same analyte or different analytes, depending on the regeneration process applied. FIG. 21 is an example workflow for surface regeneration of a patterned substrate formed by any of the disclosed techniques. Substrate manufacture (block 1200) involves patterning of a top layer of a (see FIGS. 14-16) to reveal or expose binding sites on a binding layer. Activation (block 1202) involves creation of two different reactive groups on the binding sites and the interstitial or remaining top layer, e.g., a metal oxide layer. This can be achieved using plasma or other chemical treatment. Passivation (block 1204) of the interstitial region involves deposition of polymer exclusively on the interstitial surface while leaving the binding sites unmodified.

DNA origami placement (block 1206) involves loading the binding site with the DNA origami (e.g., a core structure 13 as provided herein), that can carry with it one or more unique anchor molecules onto which other molecular cargos can be tethered. These unique anchor molecules could be single stranded DNA, thiol, amine, azide, DBCO etc. Cargo loading (block 1208) is the process by which one or more unique molecules can be loaded onto the surface organized DNA origami, e.g., via linkages to the unique anchor molecules. The unique molecules of the cargo loaded onto the DNA origami define the assay functionality of the substrate at the binding sites. In one example, the cargo includes or binds to one or more analytes in a sample. The unique molecules, in an embodiment, may include capture and detector molecules. The assay is performed (block 1210) to assess binding at the DNA origami for quantifying the number of analytes in a sample. The sample storage (block 1212) involves optionally preserving the substrate after the assay has been performed on it.

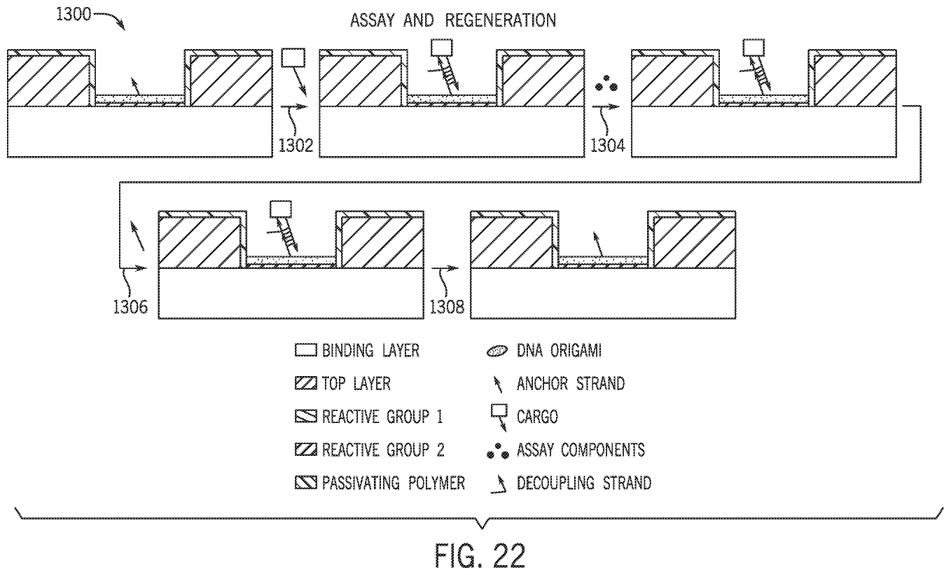

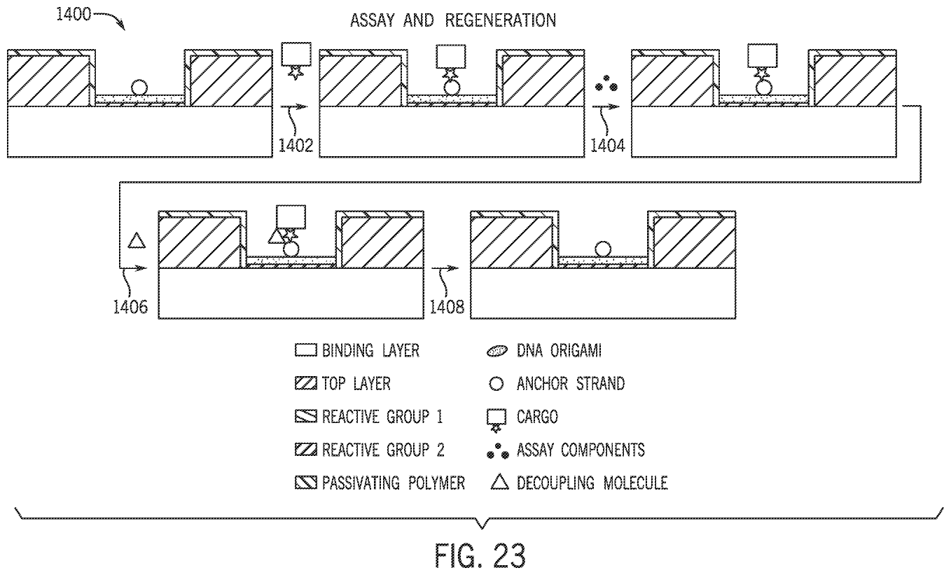

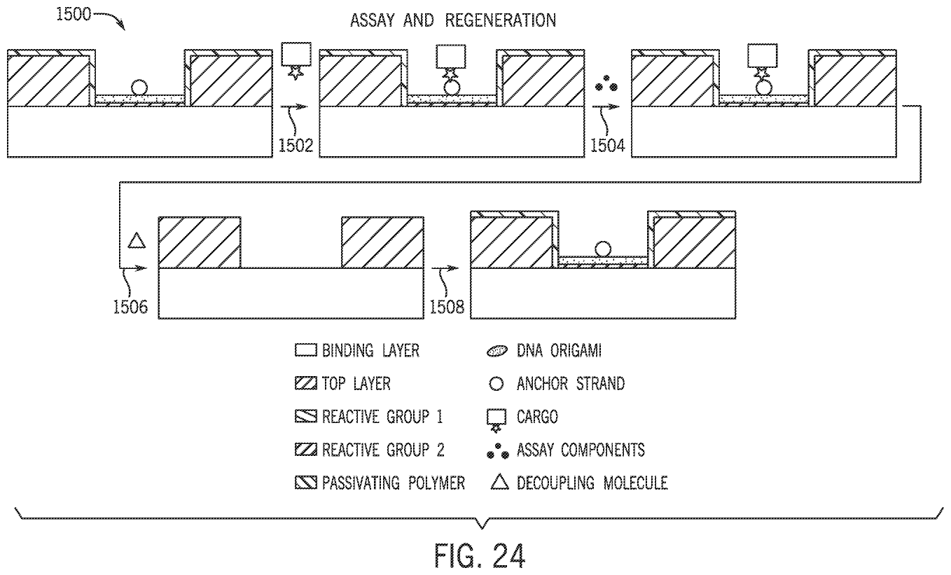

The first approach to regeneration involves removal of the cargo after the assay performance (block 1230). Example cargo removal protocols are shown in FIGS. 22-23. The second approach to regeneration involves removal of the DNA origami itself (block 1240) using an example protocol shown in FIG. 24. It should be understood that the substrates disclosed herein may be regenerated using cargo removal and/or DNA origami removal. Further, the same substrate can be subjected to repeated assay and regeneration steps of the first and/or second type

FIG. 22 shows an example cargo removal procedure 1230 as in FIG. 21. The procedure may be performed or initiated at step 1300 on a substrate, such as a patterned substrate formed as shown in FIGS. 14-17. In the illustrated example, the substrate has an unloaded DNA origami associated with each binding site on the binding layer. Step 1302 illustrates the incubation of the cargo on the substrate with DNA origami to enable a single cargo element to be loaded onto the DNA origami using a unique anchor site as a docking location. The cargo could be a single molecule or a group of molecules. There could be single cargo on each DNA origami or a plurality of molecules. Step 1304 illustrates the addition of assay components that interact with the cargo, resulting in a unique signal being generated. After the performance of the assay, in step 1306, a unique decoupling molecule is added to the substrate at step 1308 to decouple the cargo molecule from the DNA origami to result in a substrate that returns to step 1300 with an unloaded DNA origami associated with the binding site.

FIG. 23 shows an example cargo removal procedure 1230 as in FIG. 21. The procedure may be performed or initiated at step 1400 on a substrate, such as a patterned substrate formed as shown in FIGS. 14-17. Step 1402 illustrates the incubation of the cargo on the substrate with DNA origami to enable a single cargo element to be loaded onto the DNA origami using a ssDNA on the origami as an anchor. The cargo could be a single molecule or a group of molecules. There could be single cargo on each DNA origami or a plurality of molecules. Step 1404 illustrates the addition of assay components that would interact with the cargo, resulting in a unique signal being generated. After the performance of the assay, in step 1406 a unique ssDNA is added to the solution at step 1408 that would interact with the cargo molecule and decouple it from the DNA origami through a process known as DNA strand displacement, resulting in a substrate that resembles the substrate that returns to step 1400 with an unloaded DNA origami associated with the binding site.

FIG. 24 shows an example cargo removal procedure 1240 as in FIG. 21. The procedure may be performed or initiated at step 1500 on a substrate, such as a patterned substrate formed as shown in FIGS. 14-17. Step 1502 illustrates the incubation of the cargo on the substrate with DNA origami to enable a single cargo element to be loaded onto the DNA origami using a unique anchor site as a docking location. The cargo could be a single molecule or a group of molecules. There could be single cargo on each DNA origami or a plurality of molecules. Step 1504 illustrates the addition of assay components that would interact with the cargo, resulting in a unique signal being generated. After the performance of the assay, in step 1506 the substrate is treated with an extremely reactive solution which could be a strong acid (pH<2) or a strong base (pH>10) that could be used to remove all the organic molecules from the substrate including the cargo molecule, DNA origami as well as passivating polymer layer. Subsequently, the method performs the steps shown, for example, in FIGS. 14-17, to form a patterned substrate at step 1508 to create a surface with a single origami bound to each binding site. As provided herein, the DNA origami can be a loaded or unloaded origami molecule.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-52. (canceled)

53. A method for forming a substrate for detection of an analyte molecule in a sample, the method comprising:
providing a base layer;
providing a binding layer on the base layer;
depositing a top layer on the binding layer;
patterning the top layer to expose portions of the binding layer, the exposed portions corresponding to a plurality of binding sites on the binding layer; and
providing a supramolecular structure associated with each binding site of the plurality of binding sites, the supramolecular structure comprising:
a core structure comprising a plurality of core molecules
a capture molecule linked to the core structure at a first location, and
a detector molecule linked to the core structure at a second location, wherein the supramolecular structure is in an unstable state, such that the detector molecule is configured to be unbound from the core structure through cleavage of a link therebetween at the second location and such that the respective capture molecule and detector molecule spaced apart are at a pre-determined distance in the unstable state and wherein, in a stable state, the detector molecule and the capture molecule are linked together through binding to the analyte molecule, thereby forming a link between the detector molecule and capture molecule, wherein the detector molecule remains linked to the core structure through the link with the capture molecule and such that the analyte molecule is associated with the individual binding site.

54. The method of claim 53, comprising activating the patterned top layer and depositing a passivating layer on the activated top layer, wherein the passivating layer is not reactive with the exposed portions of the binding layer.

55. (canceled)

56. The method of claim 54, wherein the passivating layer is a polymer layer reactive with reactive group on the top layer formed by the activating.

57. The method of claim 53, comprising activating exposed portions of the binding layer to form the plurality of binding sites.

58. The method of claim 58, wherein activating the exposed portions of the binding layer comprises plasma or chemical treatment.

59. (canceled)

60. The method of claim 59, wherein the wells are between 5 angstroms and 300 nm in depth.

61. The method of claim 53, wherein depositing or growing a binding layer on the base layer further comprises:
protecting a portion of the binding layer with a sacrificial film;
etching or removing an unprotected portion of the binding layer to form a patterned surface in the binding later; and
removing the sacrificial film to expose the patterned surface in the binding layer, wherein the top layer is deposited on the patterned surface.

62. The method of claim 53, wherein the binding layer comprises silicon, silicon dioxide, silicon nitride, graphene, quarts, gold, silver, metal, platinum, palladium, PDMS, or a polymer film.

63. The method of claim 53, wherein the top layer comprises a metal oxide, graphene, HfO₂, or CO₂.

64. The method of claim 53, wherein the base layer comprises a planar support.

65. The method of claim 53, comprising coupling the substrate to a detection system that detects a shift of the supramolecular structure from the unstable state to the stable state at each binding site of the plurality of binding sites.

66.-67. (canceled)

68. The method of claim 53, wherein providing the supramolecular structure associated with each binding site of the plurality of binding sites comprises associating an individual core structure with an individual binding site of the plurality of binding sites and, subsequent to the associating, linking the capture molecule to the core structure at the first location and linking the detector molecule linked to the core structure at the second location.

69. The method of claim 53, comprising removing the supramolecular structure from each binding site to regenerate the substrate.

70. (canceled)

71. The method of claim 70, wherein providing the supramolecular structure associated with each binding site of the plurality of binding sites comprises associating an individual core structure with an individual binding site of the plurality of binding sites and, subsequent to the associating, linking the capture molecule to the core structure at the first location and linking the detector molecule linked to the core structure at the second location.

72. A substrate for detecting one or more analyte molecules in a sample, the substrate comprising:
a base layer;
a binding layer on the base layer;
a patterned top layer exposing portions of the binding layer, the exposed portions corresponding to a plurality of binding sites on the binding layer; and
a supramolecular structure associated with each binding site of the plurality of binding sites, the supramolecular structure comprising:
a core structure comprising a plurality of core molecules,
a capture molecule linked to the supramolecular core at a first location, and
a detector molecule linked to the supramolecular core at a second location, wherein the supramolecular structure is in an unstable state, such that the detector molecule is configured to be unbound from the core structure through cleavage of a link therebetween at the second location and wherein each supramolecular structure is configured to shift from the unstable state to a stable state through interaction between the detector molecule, the capture molecule, and a respective analyte molecule of the one or more analyte molecules.

73. The substrate of claim 72, comprising a passivating layer disposed on the top layer and not on the binding layer.

74. The substrate of claim 73, wherein the passivating layer is a polymer layer reactive with reactive group on the top layer formed by the activating.

75. The substrate of claim 72, wherein the top layer has a prescribed thickness and forms a plurality of wells in which the respective plurality of binding sites are disposed, wherein walls of the wells are formed by material of the top layer.

76. (canceled)

77. The substrate of claim 72, wherein the binding layer comprises silicon, silicon dioxide, silicon nitride, graphene, quarts, gold, silver, metal, platinum, palladium, PDMS, or a polymer film.

78. The substrate of claim 72, wherein the top layer comprises a metal oxide, graphene, HfO₂, or CO₂.

79. The substrate of claim 72, wherein the base layer comprises a silicon wafer.

80. The substrate of claim 72, wherein the substrate is coupled to a detection system that detects a shift of the supramolecular structure from the unstable state to the stable state at each binding site of the plurality of binding sites.

81.-82. (canceled)

83. The substrate of claim 72, wherein each core structure independently comprises a scaffolded deoxyribonucleic acid (DNA) origami, a scaffolded ribonucleic acid (RNA) origami, a scaffolded hybrid DNA:RNA origami, a single-stranded DNA tile structure, a multi-stranded DNA tile structure, a single-stranded RNA origami, a multi-stranded RNA tile structure, hierarchically composed DNA or RNA origami with multiple scaffolds, a peptide structure, or combinations thereof.

84. A method for forming a substrate, the method comprising:
providing a base layer;
providing a binding layer on the base layer;
depositing a top layer on the binding layer;
patterning the top layer to expose portions of the binding layer, the exposed portions corresponding to a plurality of binding sites on the binding layer; and
providing a supramolecular structure associated with each binding site of the plurality of binding sites, the supramolecular structure comprising a core structure, the core structure comprising a plurality of core molecules.

85. The method of claim 84, wherein the core structure comprises comprises a scaffolded deoxyribonucleic acid (DNA) origami, a scaffolded ribonucleic acid (RNA) origami, a scaffolded hybrid DNA:RNA origami, a single-stranded DNA tile structure, a multi-stranded DNA tile structure, a single-stranded RNA origami, a multi-stranded RNA tile structure, hierarchically composed DNA or RNA origami with multiple scaffolds, a peptide structure, or combinations thereof.

86. The method of claim 84, wherein the core structure interacts with the binding layer to form one or more salt bridges to associate the supramolecular structure with each binding site.

87. The method of claim 86, comprising linking a capture molecule linked to the core structure at a first location, and linking a detector molecule linked to the core structure at a second location.

88. The method of claim 87, wherein the linking is performed after the salt bridges are formed.

89. (canceled)
